SEQUENCE LISTING (37 sequences)

SEQ ID NO: 1 - DNA, 18 bp, Desulfovibrio vulgaris
cggcgccgttgcccaggg

SEQ ID NO: 2 - DNA, 18 bp, Desulfovibrio vulgaris
tctgttcgaagagtgggg

SEQ ID NO: 3 - DNA, 18 bp, Desulfovibrio vulgaris
ttcaaggacggttacggc

SEQ ID NO: 4 - DNA, 18 bp, Desulfovibrio vulgaris
cttcaagcacggttacgg

SEQ ID NO: 5 - DNA, 18 bp, Desulfovibrio vulgaris
  misc_feature: n=A, C, G, or T h=A, C, or T
tcngccathaayacntac

SEQ ID NO: 6 - DNA, 18 bp, Desulfovibrio vulgaris
gcagatcatgatcaacgg

SEQ ID NO: 7 - DNA, 18 bp, Desulfovibrio vulgaris
gggccgtaaccgtccttg

SEQ ID NO: 8 - DNA, 18 bp, Desulfovibrio vulgaris
tcacgaagcacttccact

SEQ ID NO: 9 - DNA, 18 bp, Desulfovibrio vulgaris
gcacatgtcgaggaagtc

SEQ ID NO: 10 - DNA, 18 bp, Desulfovibrio vulgaris
accggaggagaacttgtg

SEQ ID NO: 11 - DNA, 17 bp, Desulfovibrio vulgaris
gatggaaaaccgcttcg

SEQ ID NO: 12 - DNA, 17 bp, Desulfovibrio vulgaris
aagttctcctccggttc

SEQ ID NO: 13 - DNA, 17 bp, Desulfovibrio vulgaris
accatgatggaaaaccg

SEQ ID NO: 14 - DNA, 17 bp, Desulfovibrio vulgaris
tgaccatgatggaaaac

SEQ ID NO: 15 - DNA, 17 bp, Desulfovibrio vulgaris
cgaagcatcatgtggtt

SEQ ID NO: 16 - DNA, 17 bp, Desulfovibrio vulgaris
ccggaggagaacttgtg

SEQ ID NO: 17 - DNA, 17 bp, Desulfovibrio vulgaris
aggcgcatcatgaagtt

SEQ ID NO: 18 - DNA, 17 bp, Desulfovibrio vulgaris
atgtgctgcatgtgcag

SEQ ID NO: 19 - DNA, 23 bp, Desulfovibrio vulgaris
gacggttacgghcckgtyggygc

SEQ ID NO: 20 - DNA, 32 bp, Desulfovibrio vulgaris
  misc_feature (1)..(32): N=A, C, G, or T
gacggttacgghcckgtyggygcntggttcct

SEQ ID NO: 21 - DNA, 23 bp, Desulfovibrio vulgaris
  misc_feature (1)..(23): N= A, C, G, or T
gghcckgtyggygcntggttcct

SEQ ID NO: 22 - DNA, 23 bp, Desulfovibrio vulgaris
tggttcctkctsttcaargcbaa

SEQ ID NO: 23 - DNA, 24 bp, Desulfovibrio vulgaris
ctsttcaargcbaargcyaccaac

SEQ ID NO: 24 - DNA, 26 bp, Desulfovibrio vulgaris
aaccgcgcvatgctsaarccytacga

SEQ ID NO: 25 - DNA, 26 bp, Desulfovibrio vulgaris
tacgargawcgcgghtacgcmaaggg

SEQ ID NO: 26 - DNA, 1979 bp, Archaeoglobus fulgidus
cttaaaggtgaggtggtagaaatggtatattatccgaaaaagtatgagttgtataaggcagatgaagtgccgacagaggttgtggagacggacatcttgattatcggaggaggtttctccggctgtggtgcagcgtacgaggctgcctactgggcaaaggttggcggtttgaagcttacgcttgttgagaaagcagcagttgagagaagcggagctgttgcccagggtctttcagccattaacacatacatcgaccttaccggcaggtccgagaggcagaacacccttgaggattacgtcagatacgtcaccctcgacatgatgggattggcgagagaggaccttgttgctgactacgcaaggcatgttgacggaacggtccacctcttcgagaagtggggactgcccatctggaagactcccgatgggaagtacgtcaagagagggacagtggcagataatgattcacgggtgagagctacaagccaatcatcgcaggaagctggaaggatggaagtcggtaaggaggaacatctacgagagagtttttcatcttccgagcttctgaagggcaagaacgaccccaacgctgtggccggagccgtcggtttcagcgttagagagcccaagttctacgtgttcaaggcgaaagccgtcattctggcaaccggaggtgcaacactgctcttcaggccgagaagcactggcgaagcagcaggaaggacatggtatgcaatcttcgacactggcagcggttactacatgggcttgaaggccggagcgatgctcacgcagtttgaacaccgcttcatacccttcaggttcaaggacggttacggcccagttggagcatggttcctgttcttcaagtgtaaggccaagaacgcgtatggagaggagtacatcaagacaagggctgcagagcttgagaagtacaagcccatcggtgcagcccagccaatcccgacaccgctgagaaaccaccaggtcatgctcgaaatcatggacggcaaccagccaatctacatgcacactgagaggcttctcgctgagctggctggaggagacaagaagaagctgaagcacatctacgaggaggctttcgaggacttcctcgacatgacagtcagccaggctctgctgtgggcctgccagaacatcgacccgcaggagcagccgtctgaagctgcaccggctgagccctacatcatgggttcacacagcggtgaggcaggtttctgggtatgcggtcctgaggatctgatgccggaggagtacgcaaagctcttcccgctgaagtacaacaggatgaccacagtcaagggactcttcgccatcggtgactgtgctggtgccaacccgcacaagttctccagggttcgttcactgaggcaggattgtagcgaaggcgcagtgatgttcatccccgagcagaagcccaacccagaaattgacgatgcggtcgttgaggaactcaagaagaaggcctacgcaccgatggagaggttcatgcagtacaaggacctctcaactgccgatgacgtcaagccagagtacatcctgccgtggcagggtcttgtcaggctgcagaagatcatggacgagtatgctgctggaattgcaacaatctacaagaccaacgagaagatgctgcagagagctcttgagctgctggccttcctgaaggaggacctcgagaagctcgctgcaagagacctccacgagctgatgagagcatgggagcttgtccacagagtctggactgctgaggcacacgtcaggcacatgctcttcagaaaggaaaccagatggcccggatactactacagaaccgactacccagagctcaacgacgaggagtggaagtgcttcgtctgcagcaagtacgacgctgagaaggacgagtggaccttcgagaaagtgccgtacgtgcaggtcatcgagtggagcttctaaagctctaaaatttttcttttttc

SEQ ID NO: 27 - DNA, 1531 bp, Desulfovibrio termitidis
  misc_feature (1)..(1531): n=A, C, G, or T
agagtggggcctgccctgctggatcaagaaggacggcaagaacctcgacggcgccaaggctaaggctgaaggcctggccctgcgcaacggcgactccccggtccgttccggtcgctggcagatgatgatcaacggtgagtcctacaagtgcatcgtggcagaagctgccaagaacgcactgggcgaagaccgttacatggagcgcatcttcatcgttaagatgctgctggacgccaacgagcccaaccgcatcgccggtgccgtcggcttctccacccgtgaaaacaaggtctactacttccgctgcaacgctgctgctgttgcctgcggtggtgccgttaacgtgtaccgcccccgctccaccggtgagggtatggtcgcgcttggtaccccgtctggaacgccggctccacctacaccatggtggctcaggttggcggcgaaatgaccatgatggaaaaccgcttcgtccccgcccgcttcaaggacggttacggaccggtcggcgcttggttcctgctcttcaaggcgaaagccaccaactacaagggcgaagactactgcgaaaccaaccgcgccatgctcaagccctacgaagatcgcggctacgccaagggtcacatcatcccgacctgcctgcgtaaccacatgatgcttcgcgaaatgcgcgaaggtcgtggtccgatctacatggacaccaagactgcgctgctgaacacggtcaacaacgacctgaccagccccgagtggaagcacctcgagtccgaagcctgggaagacttcctcgacatgtgcgtcggccaggccaacctctgggccgccaccaactgcgctccggaagaccgcggctccgaaatcatgccgactgaaccctacctcctcggctcccactccggttgctgcggtatctgggtttccggtccggatgaagactgggtccccgaagagtacaagatcaaggctgacaacggtaaggtctacaaccgcatgacctccgtcaacggcctctggacctgtgctgacggtgttggcgcctccggtcacaagttctcctccggttcccacgctgaaggccgcatcgtcggtaagcagatggtccgctgggttgttgaccacaaggacttcaagcccactccgaaggaaaacgctgccgacctcgccaaagagatctaccagccgtactacacctacctcgagggtaaggacatctccaccgacccggtggtgaacccgaactacatcaccccgaagaacttcatgatgcgcctcatcaagtgcaccgatgaatacggtggtggtgttgctaccctctacatgacttccaaggctctgctgaacaccggcttctggctgctcggcatgctcgaagaagactccaagaaaatggccgctcgcgacctgcacgaactgatgcgctgctgggagcagttccaccgcctgtggactgtccgcctgcacatgcagcacatcgagttccgcgaagaatcccgctacccgggcttctactaccgcggcgacttcatgngtctcg

SEQ ID NO: 28 - DNA, 1436 bp, Desulfotomaculum nigrificans
  misc_feature (1)..(1436): n=A, C, G, or T
cctggccctgcgcaacggngactccccggtccgttccggtcgctggcagatgatgatcnacggtgagtcctacangtgcatcgtggnagaagctgccaagaacgcactgggcgaagaccgttacatggagcgcatcttcatcgttaagatgctgctggacgccaacgagcccaaccgcatcgccggtgccgtcggcttctccacccgtgaaaacaaggtctactacttccgctgcaacgctgctgtcgttgcctgcggtggtgccgttaacgtgtaccgcccccgctccaccggtgagggtatgggtcgcgcttggtaccccgtctggaacgccggctccacctacaccatggtggctcaggttggcggcgaaatgaccatgatggaaaaccgcttcgtccccgcccgcttcaaggacggttacggaccggtcggcgcttggttcctgctcttcaaggcgaaagccaccaactacaagggcgaagactactgcgaaaccaaccgcgccatgctcaagccctacgaagatcgcggctacgccaagggtcacatcatcccgacctgcctgcgtaaccacatgatgcttcgcgaaatgcgcgaaggtcgtggtccgatctacatggacaccaagactgcgctgctgaacacggtcaacaacgacctgaccagccccgagtggaagcacctcgagtccgaagcctgggaagacttcctcgacttgtgcgtcggcnaggccaacctctgggccgccaccanctgcnctccggaagaccgcggctccgaaatcatgccnactgaaccctacctcctcggctcccactccggttgctgcggtntctgggtttccggtccggatgaanactgggtccccgaagagtacaagatcgaggctgacagcggtaaggtctacaancgcatgacctccgtcaacggcctctggacctgtgctgacggtgttggcgcctccggtcacaagttctcctccggttcccacgctgaaggccgcatcgtcggtaagcagatggtccgctgggttgttgaccacaaggacttcaagcccactcngaaggaaaacgctgccgacctcgccaaagagatctaccanccgtactacacctacctcgagggtaaggacatctccaccgacccggtggtgaacccgaactacatcaccccgaagaacttcatgatgcgcctcatcaagtgcaccgatgaatacggtggtggtgttgntaccctctacatgacttccaaggctctnctgaacaccggcttctggctgctcgncatgctcgaagaagactccaagaaaatggccgctcgcgacctgcacgaactgatgcgctgctgggagcagttccaccgcctgtggactgtccgcctgcacatgcagcacatggagttccgcgaagnatcccgctacccgggcttctact

SEQ ID NO: 29 - DNA, 2097 bp, Desulfovibrio vulgaris
ggcggcaaccgctaagcggacaacaacgcaacttggtgcgataaggagattaaatcatgccgatgattcccgttaaggaacagccgaagggtgttgccatcgccgaaccgaccgtgaaggaacatgatgttgaccttctcatcgtcggtggcggcatgggcgcgtgcggtaccgctttcgaagccgtccgctgggccgacaagtacgctcctgaactgaagatcctgctgatcgacaaggcctccctcgagcgctccggcgccgttgccccgggcctgtccgccatcaatacctaccttggtaagaacgacgccgacgactacgtccgcatggttcgtaccgacctcatgggcctcgttcgcgaagacctcatcttcgaccttggccgtcacgtcgacgactccgtccatctgttcgaagagtggggcctgccctgctggatcaaggacgagcatggtcacaacctcgacggtgcccaggccaaggccgctggcaagtcgctccgcaacggcgacgacccggtccgctccggtcgttggcagatcatgatcaacggtgaatcctacaagtgcatcgtcgccgaagctgcgaagaacgcccttggtgaagcccgactcatggagcatcttcatcgtgaagctgctgctcgacgccaacacccccaaccgcgtggctggcgccgtgggcttcaacctgcgcgccaacgaagtgcacatcttccgctccaacgccatgctggttgcctgtggcggcgcggtcaacgtgtacaagccccgctccaccggtgaaggcatgggccgtgcatggtaccccgtgtggaacgccggttcgacctacaccatgtgtgcccaggtcggcgccgaaatgaccatgatggaaaaccgcttcgtccccgcccgcttcaaggacggttacggcccggttggcgcatggttccttctgttcaaggcgaaggccaccaactacaagggtgaagactactgcgccaccaaccgcgcaatgctcaagccctacgaagaccgcggctacgccaagggccacgtcatcccgacctgcctgcgtaaccacatgatgcttcgcgaaatgcgcgaaggtcgtggtcccatctacatggacaccaagaccgccctgcagtccaccttcgcgaacatgacccccgagcagcagaagcacctcgagtccgaagcttgggaagacttcctcgacatgtgcgtgggtcaggccaacctctgggcttcgatgaacatccagcccgaagagcgcggttctgaaatcatgcccaccgagccttacctgctcggttcgcactccggttgctgcggtatctgggtttccggtcccgacgagaagtgggtgcccgaagactacaaggtgcgcgcttccaacggcaagatctacaaccgcatgaccaccgtcgaaggtctgtggacctgcgctgacggcgttggcgcctccggccacaagttctcctccggttcgcacgccgaaggccgtatctgcggcaagcagatggtccgctggtgcctcgaccacaaggattacaagcccgccatcaaggaaagcgcggacgagctggtgaagctcatctaccgtccgtactacaactacatggaaggcaaggccgcttcgaccgaccccgtggtgaacccgtcctacatcacgcccaagaacttcatgatgcgcctcgtgaagtgcaccgacgaatacggcggtggcgtgggtacctactacaccacttcggccgcggctcttgatacgggcttcagcctcctcggcatgctcgaagaagactcgctgaagctggccgctcgcgacctgcacgaactgctccgctgctgggaaaactaccatcgcctgtggaccgtgcgcctgcacatgcagcacatccgcttccgcgaagagtcccgttaccccggcttctactaccgcgccgacttcatgggtctggacgactccaagtggaagtgcttcgtgaactcgaagtacgatcccgccactggcgagaccaagatcttcaagaaggcctactaccagatcatccccgaataggatgagcaccagggcggttgcggggtaccgcaaccgcccttttcactt

SEQ ID NO: 30 - DNA, 1436 bp, Thermodesulfobacterium mobile
  misc_feature (1)..(1436): n=A, C, G, or T
tgtccctgcgcancgncgncaatcntgtccgctccggccgctggnagatcatgatcaacggnnantcctacnantgcatcgtngncgaggctgcnaaaaacgccctgggccaggancgcntcatggatcgnntcttcatcgtgaagctgctcctcgacgccnancancncanccgcatcgccggtgcngtcggcttcttccncccnnnaaaacntagtgttcntcttcnnngccaacgccatcctggtggcctgcggcggcgcngtcaacgtgtaccgcccccgctccaccggtganggcatgggccgcncctggtacccngtctggaacgctggttccacctacaccatgtgngctnnngtcngcgccnanatgacnatgatngaaaaccgcttcgtccccgcccgcttcaaaganngttacngcccggtcggcgcttggttcctgctgttcaggctaangccaccaacttccgnnngtgaagactactgcgcgaccaacagngccatgctgaagccctacgangatcgcggctacgccaagggtcacgtcatccccacctgcctgcgtaaccacatgatgctccgtgaaatgcgtgaaggtcgcggtcccatctacatggacaccaagacngccctgctgagcaccttcganaccntgncncccgnacancagaagcacctcgagtccnaagcnngggaagacttcctcgacatgtgcntnggtcaggctnacctctgggccnncatnaacntncngccngaagaagtnggttctgaaatcatgcccaccgagccntacctgctcggttcncactccggntgctgcggnatctgggnttccggtcccgacgaataatgggtgcccgangactacnanatcnncgccgagaacggcaaggtctacnaccgtatgaccaccgtngaaggcctgtngacctgcgctgacggcgtaggcgcttccggtcacaagttctcctccggttcgcacgccgaaggtcgtatctgcggtaagcagatggtccgctggnncntcgaccacaaggattncaagccggcnatnnnngaaanggntgaaganctggccaaagngatctaccncccntagtacacctacgngganggcaaggacgtttccacngacccngtggtgaacccggagtacatcactccnnagaacttcatgatgcgtctggtgaagnncaccgacgaatacggcggcggngtnnncacctagtacacnacctcccaggctgntctggacaccggcttccacctgctggacatgctggaagaagactccctnaagctggctgcccgtgacctgcacganctgatncgctgctgggaacagttccaccgcctgtggaccgttcgnctgcacatgcagcacatcgcnttccgcgaagaatnccgttacccnggcttctactaccg

SEQ ID NO: 31 - DNA, 1497 bp, Desulfomicrobium baculatum
aagaaagacggcaagaacctcgacggcgcacaggccaagaaagagggcatgtccctgcgcaccggcgctgctcctgtccgctccggccgttggcagatcatgatcaacggtgagtcctacaaggtcatcgttgctgaagccgccaagaacgctctgggttccgaccgttacatggagcgcatcttcatcgttaagctgctgcttgatgccaaggttccgaaccagatcgccggcgcagtcggtttctccgttcgtgaaaacaaagtgtacgtcatcaaggccaagaccatgtccgtggcttgcggtggcgctgttaacgtataccgtccccgctccactggtgaaggccttggtcgcgcatggtatcccgtatggaacgccggctccacctacaccatgtgtgctcaggttggcgctgaaatgaccatgatggaaaaccgcttcgtacctgcccgtttcaaagacggttacggtccggtcggcgcatggttcctgctcttcaaggccaaagccaccaacgccaagggtgaagactactgtgtcaccaaccgcgccatgctgaagccctacgaagatcgcggttacgcaaagggtcacgtcattccgacctgtctgcgcaaccacatgatgcttcgcgaaatgcgcgaaggtcgcggccccatctacatggacaccgccaccgccctgcagaccaccttcaaggaactgtccaaggccgagcagaagcatcttgagtccgaagcttgggaagacttcctggatatgtgtgttggccaggccaacctgtgggcagccatgaacatcaagcccgaagaacgcggctccgagatcatgcccaccgaaccttacctgctcggctcccactccggctgctgcggcatctgggtatccggtcctgcagagtcctgggttcctgaagaataccaggtcaaggcagccaacggaaaggtctacaaccgcatgaccacggtcaacggtctgttcacctgcgctgacggcgttggcgcttccggtcacaagttctcctccggttcccatgctgaaggccgtatcgtcggcaagcagatggttcgctacgttgtggatcacaaggatttcactcccacgctgaatctgtcctccgaagaactgaagaaggaaatctaccagccttggtacacctacgagcagttcaagggtgcttccactgatccggtagtcaacccgaactacatctcgcccaacaacttcatgatgcgcctcatcaaggccaccgatgaatacggcggcggtgttgctactctgtacaccacttccgacagactgctcgacaccggtttcggcctgctcgacatgctggaagaagactccaagaagctggctgcccgcgacctgcacgaactgctccgttgctgggaacagtaccacagactgtggaccgttcgtctgcacatgcagcacattcgtttccgtcaggaaagccgttaccccggtttctactatcgcgcagacttcatg

SEQ ID NO: 32 - DNA, 1312 bp, Thermodesulfobacterium mobile
  misc_feature (1)..(1312): n=A, C, G, or T
atcgtggctgaggctgctaaaaatgctcttaccagctatgataaggctgagatcatcgaaagatgctttatcgtaaggcctttgttggatgcaaatgataagagccgctgtgctggtgcagtaggtttttctgtcagagaaaacaagatttacatcatcaaggccaaggctacccttctttctactggtggggcagttaacattttccgtcctaggtctatagacgaaggaaagggtcgtgcctggtatcctgtatggaaccctggaactggttatgctatgtgtgctatgactggtgctaagcttgtacttatggaaaacaggtttatccctgcccgttttaaagatggttatggtcctgtgggtgcttggttcttgcttttcaaggccagagcaaccaatgcttttggtgaggactatgtagccaaacataaggatgaactcaagaagtttgctccttatggagaggcttctcacatcggtacctgtttgagaaaccatgcaatgcttatcgaaatggaacagggtccgtggtcctatttatatgcacactgaatgggctcttcaggaagccgctgaaaagatggacnnaaaaagagttttaaacacctcattgctgaggcttgggaagactccttagacatgtgtgtaacccaaggctggattgtgggcttgtttgaaacatcgaacctgaaaattgcctttctgaaatcatgcctactgagccttatctcctcggtagccatgctggttgtgccggtgcttgggtatgcggtcctaatgaagattgggtacctgaggaatacaaggctccttggaaagaaatcggtctttacaacagaatgactactgtaaaaggtcttttctgtgctggtgacaccgttggagcttctgggcataagttctcctctggttctcacgtagaaggacgtattgcagccaaggctatggttcagtactgtcttgacaataaagactatacacctaccatcaaggaaactgctgaagagttaaagaaagagatctatggtccttggtataggtttgaagaatttaagaatacttctacccactatgaaatcaaccccaactatctcattcctcgtcatattcaggccaggcttatgaagcttatggacgaatatgtggctggtgcctctactttctacaagaccaacaagatcatgctcgagagaggtcttgaccttctcagaatgcttaaagaagacatggaatatgctgcagccagagatttgcatgaacttatgagagcttgggaaaacaggcaccgtgtatggactgctgaggctca

SEQ ID NO: 33 - DNA, 204 bp, Desulfovibrio desulfuricans
gatggaaaaccgcttcgtccccgcccgcttcaaggacggttacggtccggttggcgcttggttcctgctcttcaaggccaaagccaccaacttccgcggcgaagactactgcgtgaccaaccgcgccatgctgaagccctacgaggaacgcggctacgccaagggtcacatcatcccgacctgcctgcgtaaccacatgatgct

SEQ ID NO: 34 - DNA, 203 bp, Desulfomicrobium baculatum
gatggaaaaccgcttcgtacccgcccgtttcaaagacggttacggtccggttggcgcatggttcctcctcttcaaggccaaagccaccaacgccaagggtgaagactattgtgtcaccaaccgcgccatgctgaagccttacgaagatcgcggttacgccaagggtcacgtcatcccgacctgtctgcgcaaccacatgatgc

SEQ ID NO: 35 - DNA, 203 bp, Desulfovibrio longreachii
gatggaaaaccgcttcgtgcccgcccgcttcaaggacggttacggcccggtcggcgcgtggttcctgctgttcaaggcgaaggccaccaactacaagggtgaagactactgcgccaccaaccgcgcgatgctgaagccctacgaagatcgcggctacgccaagggtcacgtcattccgacctgcctgcgtaaccacatgatgc

SEQ ID NO: 36 - DNA, 204 bp, Desulfovibrio gracilis
gatggaaaaccgcttcgtgcccgcccgcttcaaggacggttacggcccggtcggtgcctggttcctgctcttcaaggccaaggctaccaactacaagggtgaggactactgcgagaccaaccgcgccatgctgaagccttacgaggatcgcggctacgccaagggtcacgtcatccccacctgcctgcgtaaccacatgatgct

SEQ ID NO: 37 - DNA, 302 bp, Desulfovibrio gracilis
ggatcccgaagcatcatgtggttactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatttcgaagcggttttccatcgaattc

(19) United States
(12) Patent Application Publication
(10) Pub. No.: US 2004/0091882 A1
(43) Pub. Date: May 13, 2004

(54) Method of detecting sulphate-reducing bacteria

(76) Inventors: Michel Magot, Gameville (FR); Gilles Ravot, Generac (FR)

Correspondence Address: BLANK ROME LLP, 600 NEW HAMPSHIRE AVENUE, N.W., WASHINGTON, DC, 20037, US

(21) Appl. No.: 10/347,869

(22) Filed: Jan. 22, 2003

Related U.S. Application Data
(63) Continuation-in-part of application No. 09/535,012, filed on Mar. 24, 2000, now Pat. No. 6,531,281.

(30) Foreign Application Priority Data
Mar. 24, 2000 (FR) 99 03637

(51) Int. Cl. C12Q 1/68

(52) U.S. Cl. 435/6

(57) ABSTRACT

The present invention relates to a method for the detection of sulphate-reducing bacteria in a sample which is likely to contain them, the said method comprising the extraction of the DNA or of the RNA from the said sample and the detection of at least one fragment of the APS reductase gene or at least one fragment of the mRNA transcribed from the APS reductase gene, an indicator of the presence of sulphate-reducing bacteria in the said sample.

The present invention relates to a method of detecting and enumerating sulphate-reducing bacteria.

Sulphate-reducing bacteria (SRB) use sulphate as electron acceptor under anaerobic conditions, via the anaerobic respiration of sulphates (energy reduction), to produce sulphides while recovering, during this reduction, the energy necessary for their growth This metabolic characteristic constitutes a common characteristic of these organisms, regardless of their phylogenetic position (Legall & Fauques, 1988).

These bacteria are recognized to be the principal microorganisms responsible for the biological formation of hydrogen sulphide (H₂S). This H₂S of biological origin in particular, and the metabolism of sulphate-reducing bacteria in general, cause many problems for industrialists such as the biological corrosion of steel, on the one hand, and the potential risk for staff, on the other (Postgate, 1979) In the petroleum industry, in addition to the abovementioned pernicious effects, the sulphate-reducing bacteria are also involved in impairing the quality of crude oil (Cord-Ruwich et al., 1987). The detection of these sulphurogenic bacteria therefore constitutes a major challenge for combating the production of H₂S in a large number of industrial activities (Tatnall et al., 1988).

Microbial culture techniques applied to the detection and to the enumeration of these microorganisms have been developed (API, 1982; Magot et al., 1988; Scott & Davies, 1992). These methods require an incubation time of 10 to 21 days and are therefore poorly suited to the monitoring of contaminations of fluids in real time. Alternative methods allowing rapid measurement of the level of contamination have also been developed, such as for example the “Rapid Check™” from Conocco, based on the immunodetection of APS reductase (Horacek & Gawell, 1988, EP 272,916), or the “Hydrogenase test™” (Caproco), which detects the activity of hydrogenases, enzymes which are present in the SRBs but are not specific to these organisms (Scott & Davies, 1992) However, none of these methods is sufficiently sensitive or specific.

The authors of the present invention have developed a method of detecting and enumerating sulphate-reducing bacteria which combines these two advantages: sensitivity arid specificity and which combines, in addition, speed with reliability. They therefore directed their attention to the sulphate energy reduction pathway and more specifically to that of APS reductase.

APS reductase (or Adenylylsulphate reductase) which allows the reduction of adenosine phosphosulphate (APS) (product of the activation of sulphate by ATP sulphurylase), is a cytoplasmic enzyme containing two subunits (αand β) known to be involved only in the anaerobic respiration of sulphate (Legall & Fauques, 1988) This enzyme is not therefore present in non-sulphate-reducing organisms since it is not involved in the assimilatory reduction which allows the incorporation of sulphur into various molecules necessary for life, such as amino acids and vitamins.

On the basis of two sequences of the gene encoding this enzyme deposited in data banks, one derived from an organism in the domain of Bacteria (Desulfovibrio vulgaris, em_ba. Z69372) and the other from the sector of Archaea (Archaeoglobus fulgidus: em_ba: X63435), the authors of the present invention were able to amplify and sequence various genes encoding APS reductase. Surprisingly, they observed that this gene is remarkably well conserved whereas the phylogeneric diversity of the organisms studied could not a priori suggest it. This result opened the perspective for using this gene as a target for the specific detection of sulphate-reducing bacteria, especially in the domain of Bacteria.

The subject of the present invention is therefore the use of at least one nucleotide sequence which hybridizes specifically with a fragment of the APS reductase gene or a fragment of the mRNA transcribed from the APS reductase gene to detect the presence of sulphate-reducing bacteria in a sample.

The subject of the present invention is more particularly a method for the specific, qualitative or quantitative detection of sulphate-reducing bacteria in a sample which is likely to contain them, the said method comprising the extraction of the DNA or of the RNA from the said sample and the detection of at least one fragment of the APS reductase gene or one fragment of the mRNA transcribed from the APS reductase gene, an indicator of the presence of sulphate-reducing bacteria in the said sample.

The extraction of the DNA or of the RNA from the said sample may be carried out by standard techniques which are well known to persons skilled in the art.

More particularly, the detection of at least one fragment of the APS reductase gene comprises the specific gene amplification of at least one fragment of the gene for the α subunit of APS reductase. Advantageously, the gene amplification products may, in addition, be subjected to hybridization with a probe which is specific for the said fragment of the gene for the α subunit of APS reductase, the said probe being labelled in a detectable manner.

According to another embodiment, the detection of at least one fragment of the APS reductase gene comprises the hybridization of the extracted DNA with a probe which is specific for the said fragment of the gene for the α subunit of APS reductase, the said probe being labelled in a detectable manner.

According to another embodiment, the method of the invention comprises the extraction of the RNA from a sample which is likely to contain sulphate-reducing bacteria and the detection of at least one fragment of the mRNA which is transcribed from the APS reductase gene.

In this case, the detection may be carried out by direct hybridization of a specific nucleotide probe labelled in a detectable manner with the extracted mRNA, and/or by specific amplification of the mRNA encoding APS reductase, in particular by RT-PCR (reverse transcription followed by a polymerase chain reaction).

The subject of the present invention is also an oligonucleotide having a nucleotide sequence which is essentially identical to a sequence chosen from the sequences SEQ ID No. 1 to 25. Such an oligonucleotide is in particular useful as a primer for amplifying a fragment of the gene for the α subunit of APS reductase, or as a probe which hybridizes with a fragment of the gene for the α subunit of APS reductase or the product of amplification thereof.

Preferably, it is possible to use as a primer an oligonucleotide having a sequence which is essentially identical to -one of the sequences SEQ ID No. 11 to 18, and as a probe an oligonucleotide having a sequence which is essentially identical to one of the sequences SEQ ID No. 19 to 25.

“Essentially identical” is understood to mean that the sequence of the oligonucleotide is identical to one of the sequences SEQ ID No. 1 to 25 or that it differs from one of these sequences without affecting the capacity of these sequences to hybridize with the gene for the α subunit of APS reductase. A sequence which is “essentially identical” to one of the sequences SEQ ID No. 1 to 25 may in particular differ therefrom by a substitution of one or more bases or by deletion of one or more bases located at the ends of the oligonucleotide, or alternatively by addition of one or more bases at the ends of the oligonucleotide. Preferably, such an oligonucleotide has a minimum size of 10 nucleotides, preferably of at least 14 nucleotides.

According to a preferred embodiment of the invention, the method of detecting sulphate-reducing bacteria in a sample which is likely to contain them according to the invention advantageously comprises the steps consisting in:

extracting the DNA from the said sample;

bringing the DNA extracted in step i) into contact with at least one primer consisting of an oligonucleotide having a nucleotide sequence which is essentially identical to a sequence chosen from the sequences SEQ ID No. 1 to 25, preferably No. 1 to 18, under conditions allowing the specific amplification of a fragment of the gene for the α subunit of APS reductase which may be present in the DNA extract;

bringing the product of amplification into contact with a probe consisting of an oligonucleotide having a nucleotide sequence which is essentially identical to a sequence chosen from the sequences SEQ ID No. 1 to 25, preferably No. 19 to 25, the said probe being labelled in a detectable manner, under conditions allowing the specific hybridization of the said product of amplification and the said probe;

detecting the hybridization complex formed between the product of amplification and the said probe, an indicator of the presence of sulphate-reducing bacteria in the sample.

“Conditions allowing the specific amplification” is understood to mean conditions of temperature, of reaction time and optionally the presence of additional agents which are necessary for the fragment of the gene for the α subunit of APS reductase, to which the primers as defined above have hybridized, to be copied identically.

Preferably, the amplification method used is a polymerase chain reaction (PCR) which is well known to persons skilled in the art (Sambrook et al., 1989), which uses a pair of primers as defined above.

“Conditions allowing the specific hybridization” is understood to mean high stringency conditions which prevent the hybridization of the oligonucleotide with sequences other than the gene for the α subunit of APS reductase.

The parameters defining the stringency conditions depend on the temperature at which 50% of the paired strands separate (Tm).

For the sequences comprising more than 30 bases, Tm is defined by the relationship:

Tm=81.5+0.41 (%G+C)+16.6 Log(concentration of cations)−0.63 (%formamide)−(600/number of bases)

(Sambrook et al., Molecular Cloning, A laboratory manual, Cold Spring Harbor laboratory Press, 1989, pages 9.54-9.62).

For the sequences of less than 30 bases in length, Tm is defined by the relationship:

Tm=4(G+C)+2(A+T)

Under appropriate stringency conditions, at which the a specific sequences do not hybridize, the hybridization temperature is approximately 5 to 30° C., preferably 5 to 10° C. below Tm, and the hybridization buffers used are preferably solutions with a high ionic strength such as a 6×SSC solution, for example

The oligonucleotide probes used in the method of the invention are labelled in a detectable manner. For that, several techniques are accessible to persons skilled in the art, such as for example fluorescent, radioactive, chemiluminescent or enzymatic labelling.

An internal amplification control may be advantageously used in order to avoid an ambiguous interpretation of negative results of the amplification method. Indeed, for example, an absence of amplification by PCR may be due to problems of inhibition of the reaction or to the absence of a target.

The authors of the present invention propose using, as an internal control, a plasmid including oligonucleotide sequences which allow the amplification of a fragment of the APS reductase gene, the said oligonucleotide sequences flanking a sequence differing from the said fragment of the APS reductase gene by its size and/or its sequence. The said oligonucleotide sequences which are specific for a fragment of the APS reductase gene may be chosen in particular from the sequences SEQ ID No. 1 to 25, preferably the sequences No- 11 to 1F. An example of such a plasmid is represented in FIG. 4.

Added in a limiting concentration to the PCR reaction mixture, this plasmid allows the amplification of a DNA fragment of 289 bp (base pairs), whose sequence is given in FIG. 5, when no specific target is present in the sample. Thus, in the example selected, the presence of a fragment of 289 bp, without a fragment of 205 bp, indicates the functioning of the reaction and the absence of a specific target, that is to say of sulphate-reducing bacteria from the sample studied.

The following examples and figures illustrate the invention without limiting the scope thereof.

### LEGEND TO THE FIGURES

FIG. 1 represents the alignment of the sequences of the α subunit of APS reductase which allowed the definition of the primers specific for sulphate-reducing bacteria of the Bacteria domain.

FIG. 2 represents the position of the various primers described in the examples of the α subunit of APS reductase

FIG. 3a represents a characteristic gel of an amplification with the pair of primers αsp01-αsp11.

line 1: SRB. Lane 1 and 10: molecular weight marker (100 bp, Gibco BRL); lane 2 to 9: SRL 4225 (Desulfovibrio “bastinii”); SRL 6143 (Desulfovibrio “tubi”), SRL 3851 (Desulfomicrobium baculatum); SRL 3492 (Desulfomicrobium baculatum); SRL 583 (Desulfotomaculum nigrificans); SRL 2668 (Thermodesufobacterium mobile); SRL 2801 (Thermodesulfobacterium mobile); internal control,

line 2: non-SRB. Lane 1 and 10: molecular weight marker (100 bp, Gibco BRL); lane 2 to 9: SRL 4208 (unidentified fermentative anaerobic bacterium); SRL 4207 (Dethiosulfovibrio peptidovorans) SRL 4226 (Dethiosulfovibrio peptidovorans); SRL 6459 (Thermotoga elfii); SRL 5268 (Thermoanaerobacter brockii subsp.);

SRL 7311 (Thermoanaerobacter brockii subsp. lactiethylicus) ; SRL 3138 (Thermotoga maritima); SRL 4224 (Haloanaerobacter congolense).

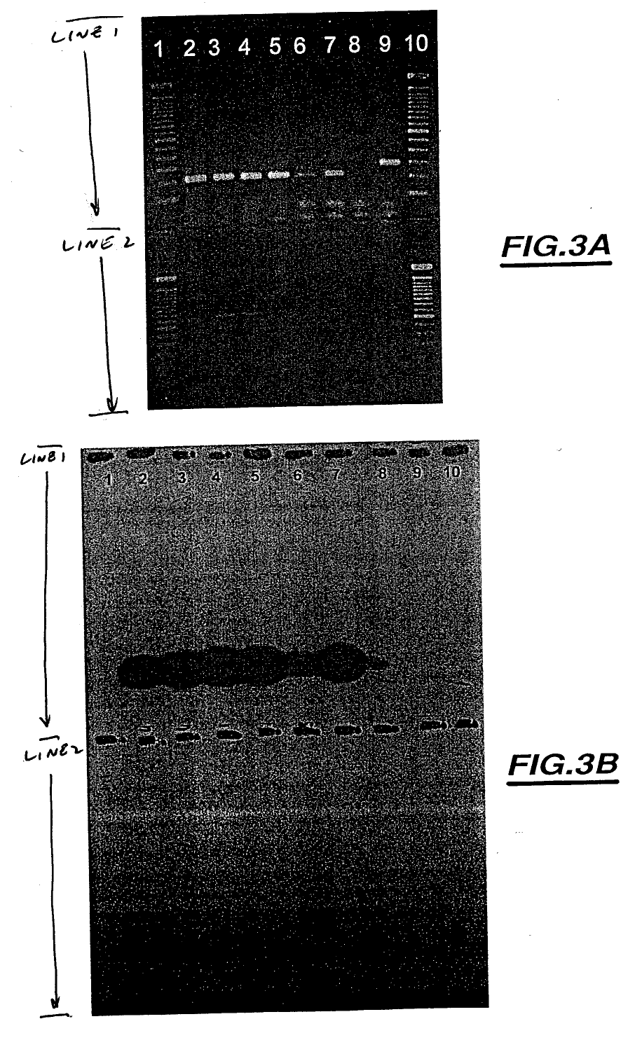

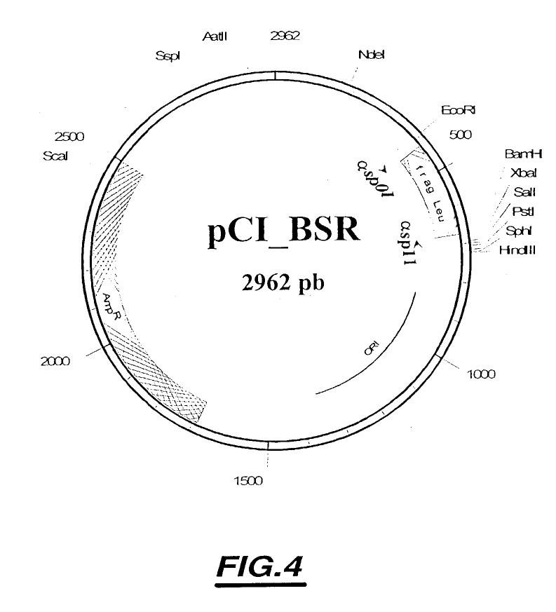

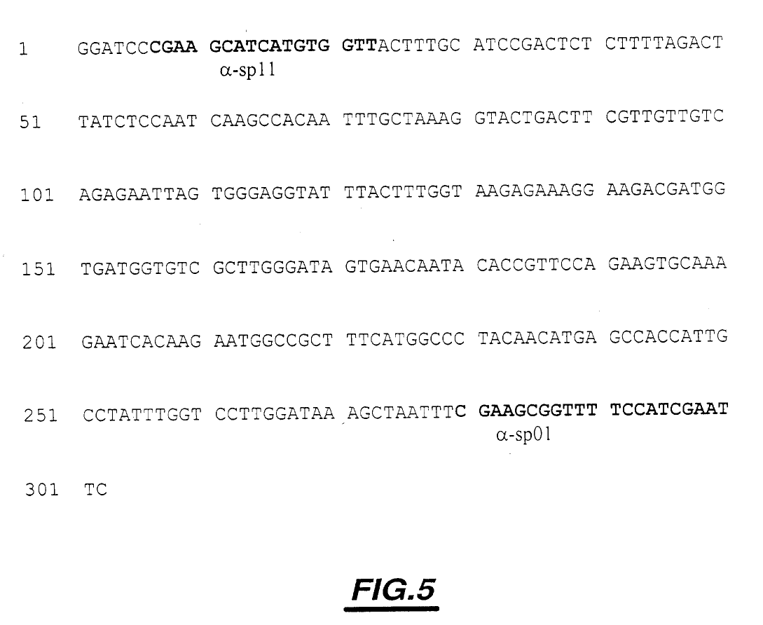

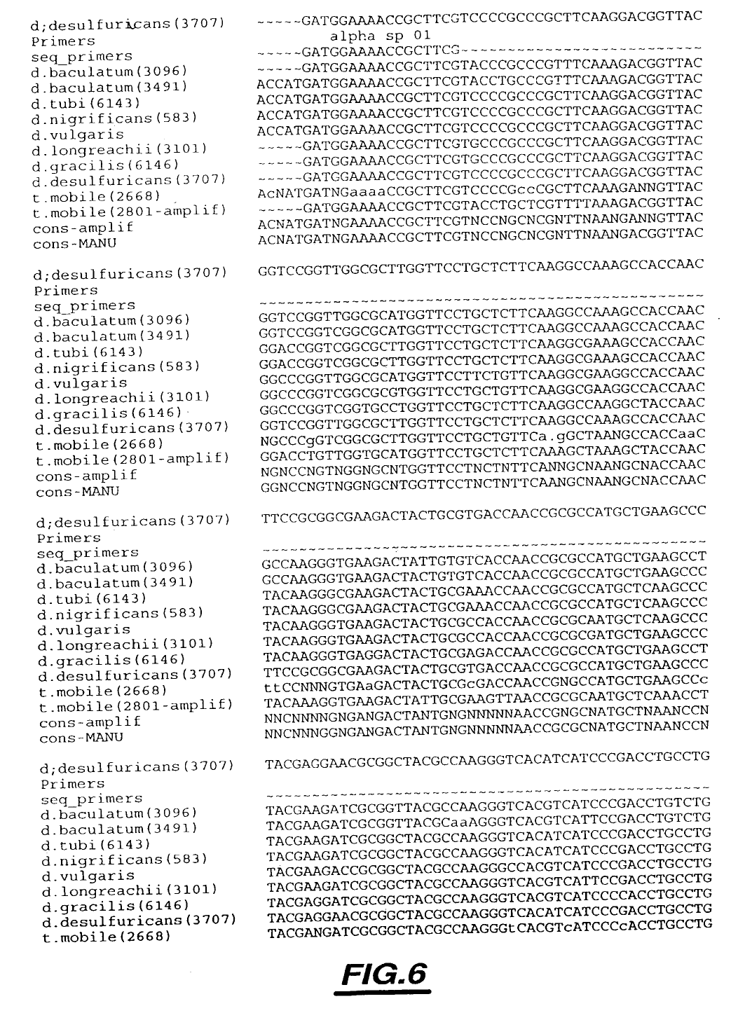

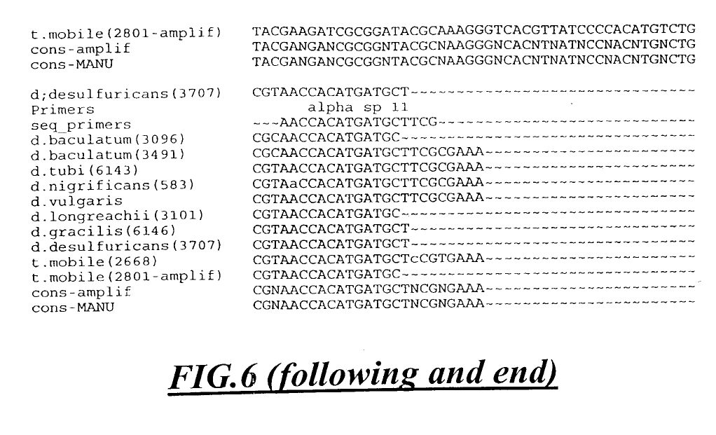

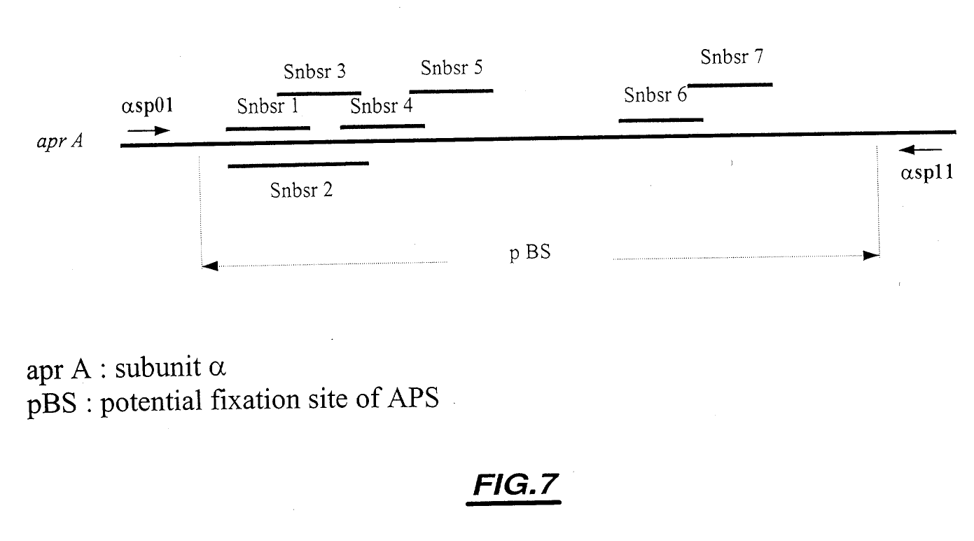

FIG. 3b represents a membrane hybridization with the oligonucleotide Snbsr 3 of the PCR products of FIG. 3a (same lanes).

FIG. 4a represents the restriction map of the plasmid pCI BSR used as an internal control, obtained from a plasmid pUC19.

FIG. 5 represents the sequence of the fragment inserted into the plasmid pCI-BSR, used as internal amplification control.

FIG. 6 represents the alignment of the fragments amplified with the primers α-sp01 and α-sp11 is which allowed the definition of the detection probes

FIG. 7 represents the position of the various oligonucleotides which hybridize specifically between the primers α-sp01 and α-sp11 in APS reductase.

### EXAMPLES

Introduction:

The experimental approach which resulted in the development of the method of the invention consists in:

1 The exploitation of the sequences of two genes for the α subunit of APS reductase which are available in data banks to construct various sets of amplification primers allowing the detection of new genes encoding this same enzyme and their sequencing.

2. The alignment of 7 sequences (2 derived from data banks and 5 obtained according to the approach detailed in point 1), making it possible to define conserved regions and to identify oligonucleotide sequences specific for this gene.

3. The evaluation of the specificity and of the sensitivity of the various sets of primers defined in point 2 on 10 strains of sulphate-reducing bacteria and 3 non-sulphate-reducing bacteria. A preferred pair of primers, leading to the amplification of a fragment of 205 bp, was validated on 36 other strains. The size of the PCR product derived from the pair of primers αsp01-αsp11 is 205 bp in the examples presented. However, it is not possible to exclude that this size varies according to the strains considered

4. The control of the specificity of this pair of primers by sequencing 5 amplified fragments of 205 bp derived from new SRBs (with respect to point 2). The 11 SRB sequences of the Bacteria domain were aligned, which makes it possible to define nucleic probes, in the conserved regions of the fragment of 205 bp, which are capable of allowing the amplification or the specific detection of all or part of the gene.

5. The evaluation of the specificity and of the sensitivity of these oligonucleotides by membrane hybridization (Southern blotting).

6. The testing on microplates of a “sandwich” type nonradioactive hybridization protocol allowing specific visualization of the product of amplification of a fragment of this gene.

7. The definition of a complete protocol allowing the detection from field samples.

8. The correlation of the results obtained by this method with those obtained by the “Kits Labège BSR™” method from field samples.

The names of bacterial species mentioned in inverted commas refer to species described in the thesis by C. Tardy-Jacquenod (1996). These species were not validated by the international committees on nomenclature because they have not yet been the subject of a publication.

### EXAMPLE 1

### Test for Sequences Encoding APS Reductase in Desulfovibrio “tubi”, Desulfotomaculum nigrificans, Desulfomicrobium baculatum and Thermodesulfobacterium mobile.

Definition of specific and conserved primers.

a) Introduction

Based on two APS reductase sequences deposited in international data banks (Archaeoglobus fulgidus: EMBL: X63435. Desulfovibrio vulgaris: EMB3L No. Z69372), the authors of the present invention were able to define several pairs of primers capable of allowing the amplification of fragments of APS reductase. These amplified fragments were able to be sequenced, allowing comparison after alignment of the various sequences Thus, the authors of the present invention were able to construct the various primers for the amplification which were specific for sulphate-reducing bacteria.

b) Materials and Methods

Sequences deposited in data bank. Two sequences encoding APS reductase are deposited in international data banks (Genbank and EMBL)

Archeoglobus fulgidus: EMBL No. X63435

Desulfovibrio vulgaris: EMBL No. Z69372

Strains Used.

SRL 7861 Acheoglobus fulgidus

SRL 4208 Non-identified fermentative anaerobic bacterium

SRL 3491 Desulfomicrobium baculatum

SRL 3096 Desulfomicrobium baculatum

SRL 583 Desulfotomaculum nigrificans

SRL 4225 Desulfovibrio “bastinii”

SRL 2840 Desulfovibrio gabonensis

SRL 3551 Desulfovibrio sp.

SRL 6143 Desulfovibrio “tubi”

SRL 4207 Dethiosulfovibrio peptidovorans

SRL 2668 Thermodesulfobacterium mobile

SRL 2801 Thermodesulfobacterium mobile

SRL 3138 Thermotoga maritima

DNA Extractions.

The extraction of the DNAs from pure strains was carried out using the kit QIAamp Tissue Kit (QIAGEN, Hilden, Germany) according to the protocol given by the manufacturer

PCR Reactions.

The primers used for this study are given in Table 1.

TABLE 1
Primers constructed for the amplification of the APS reductase gene

| SEQ ID No. | Name of the primer | Oligonucleotide sequence | Position D. vulgaris | Orientation |
|---|---|---|---|---|
| 1 | APS01 | 5′-CGGCGCCGTTGCCCAGGG-3′ | 957-914 | Sense |
| 2 | A2S02 | 5′-TCTGTTCGAAGAGTGGGG-3′ | 1110-1127 | Sense |
| 3 | APS03 | 5′-TTCAAGGACGGTTACGGC-3′ | 1603-1620 | Sense |
| 4 | APS04 | 5′-CTTCAAGGACGGTTACGG-3′ | 1602-1629 | Sense |
| 5 | APS05 | 5′-TCNGCCATHAAYACNTAC-3′ | 979-996 | Sense |
| 6 | APS06 | 5′-GCAGATCATGATCAACGG-3′ | 1239-1256 | Sense |
| 7 | APS11 | 5′-GGGCCGTAACCGTCCTTG-3′ | 1605-1624 | Antisense |
| 8 | APS12 | 5′-TCACGAAGCACTTCCACT-3′ | 2260-2677 | AntiBense |
| 9 | APS13 | 5′-GCACATGTCGAGGAAGTC-3′ | 1897-1914 | Antisense |
| 10 | APS14 | 5′-ACCGGAGGAGAACTTGTG-3′ | 2161-2178 | Antisense |

where
N: A, C, G or T
H: A, C or T
Y; C or T

The components of the gene amplification reactions (PCR) are given in Table 2.

TABLE 2
Components of the gene amplification reactions

| | | | Final |
|---|---|---|---|
| Reagents | Volumes | | concentrations |
| Sense primer (20 μM) | 1.25 | μl | 0.5 μM |
| Antisense primer (20 μM) | 1.25 | μl | 0.5 μM |
| dNTP (10 mM each)¹ | 1 | μl | 200 μM |
| PCR buffer 10X¹ | 5 | μl | MgCl₂ = 1.5 mM |
| DNA | x | μl | 2 ng/μl |
| | | | (that is 100 ng) |
| Taq DNA pol¹ | 0.4 | μl | 2 U |
| H₂O | qs 50 | μl | |

¹Boehringer Mannheim SA (Meylan, France)

PCR Programme:

| | |
|---|---|
| 1 cycle: | 3 min 95° C. |
| 5 cycles: | 1 min 94° C. |
| | 30 s X° C. |
| | T s 72° C. |
| 35 cycles: | 30 s 94° C. |
| | 30 s X° C. |
| | T s 72° C. |
| 1 cycle: | 5 min 72° C. |
| | hold 4° C. |

where T depends on the expected size of the amplified fragment: 1 min/kb

X is the hybridization temperature which depends on the pair of primers considered For a given primer

X° C. =Tm−4=4×(C+G)+2×(A+T)−4

For the analysis on a 1 or 2% agarose gel. 4 μl of PCR product were deposited, 1 μl Blue (Sambrook et al., 1989).

Sequencing of the amplified nucleic acids. The sequencing of the amplified nucleic acids was carried out directly on the crude PCR products.

Computer processing of the sequences: the Wisconsin Package programmes, version 9.1-unix, Genetics Computer Group (GCG), Madison, Wisc., were used.

c) Results

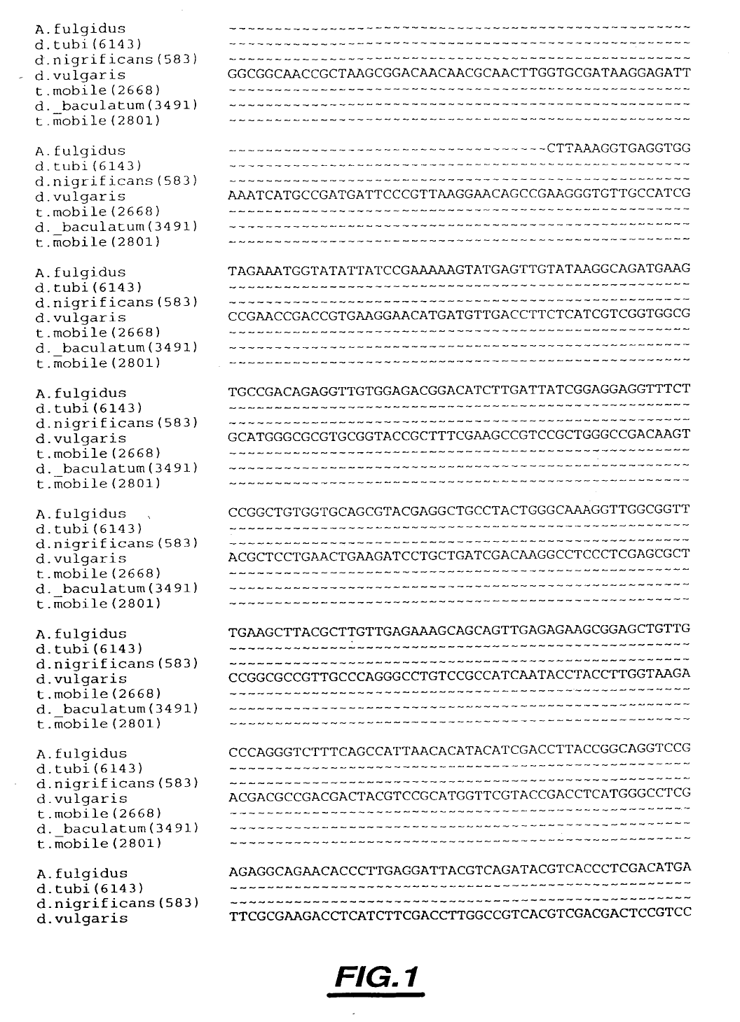

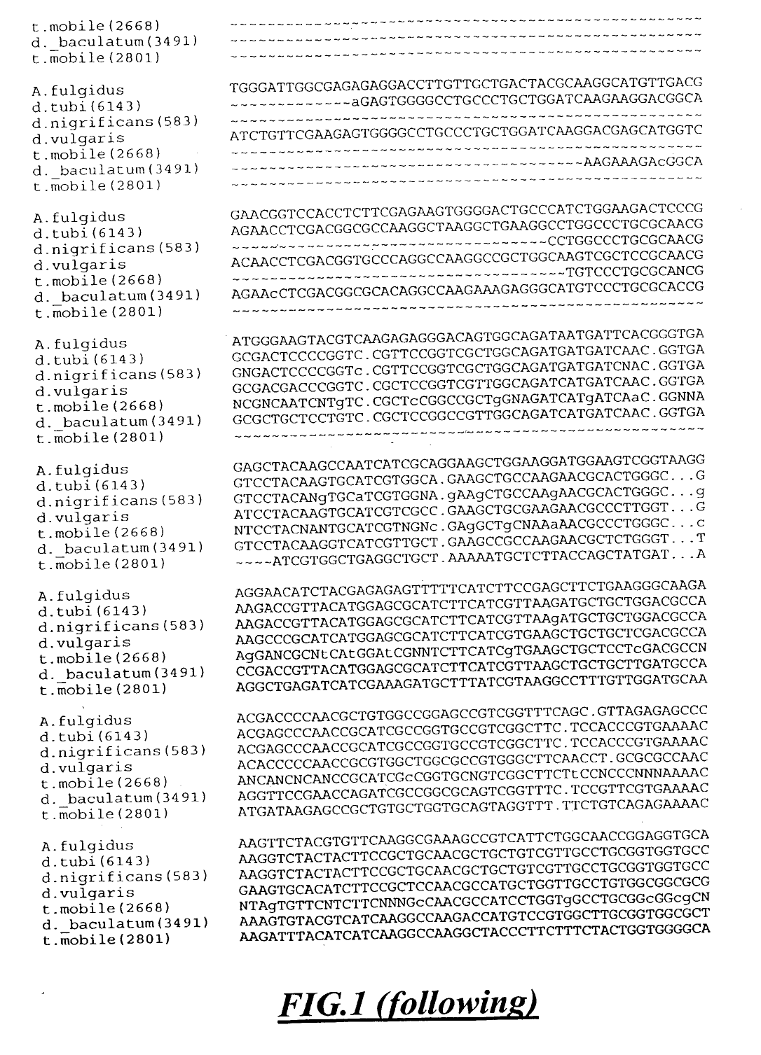

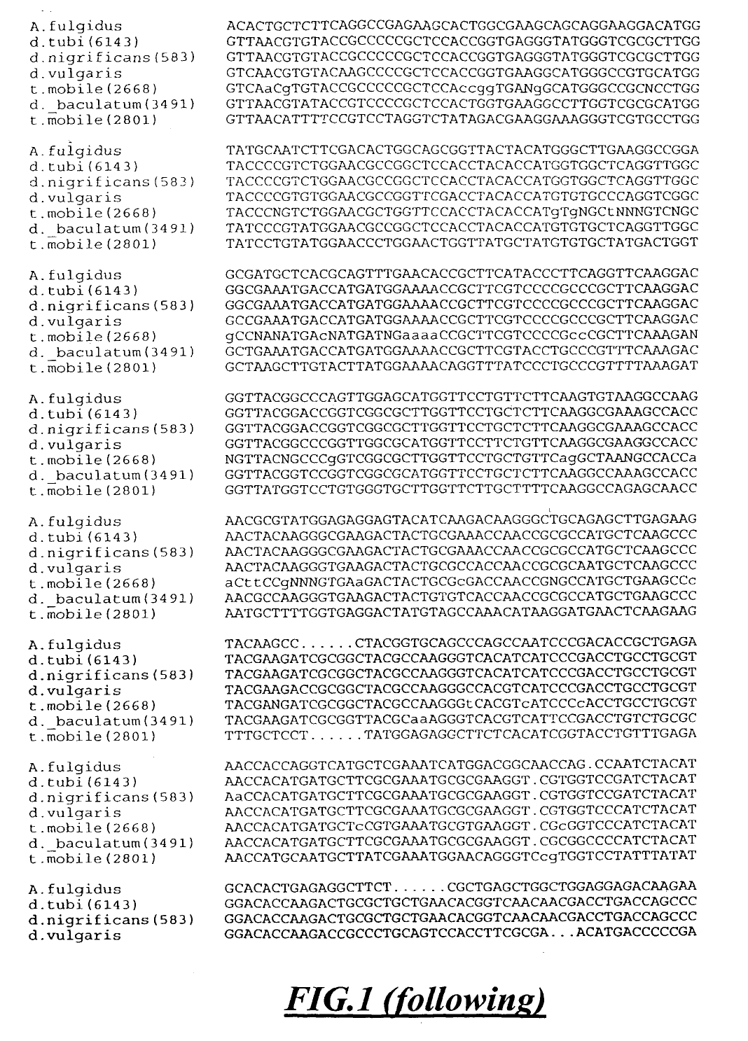

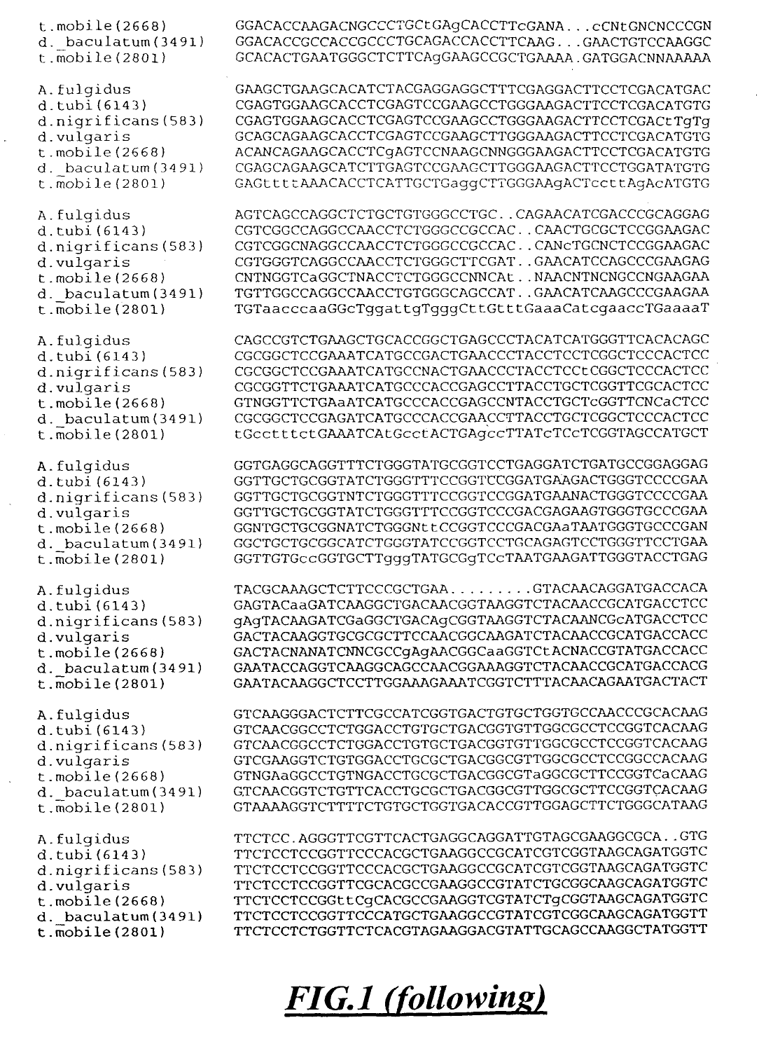

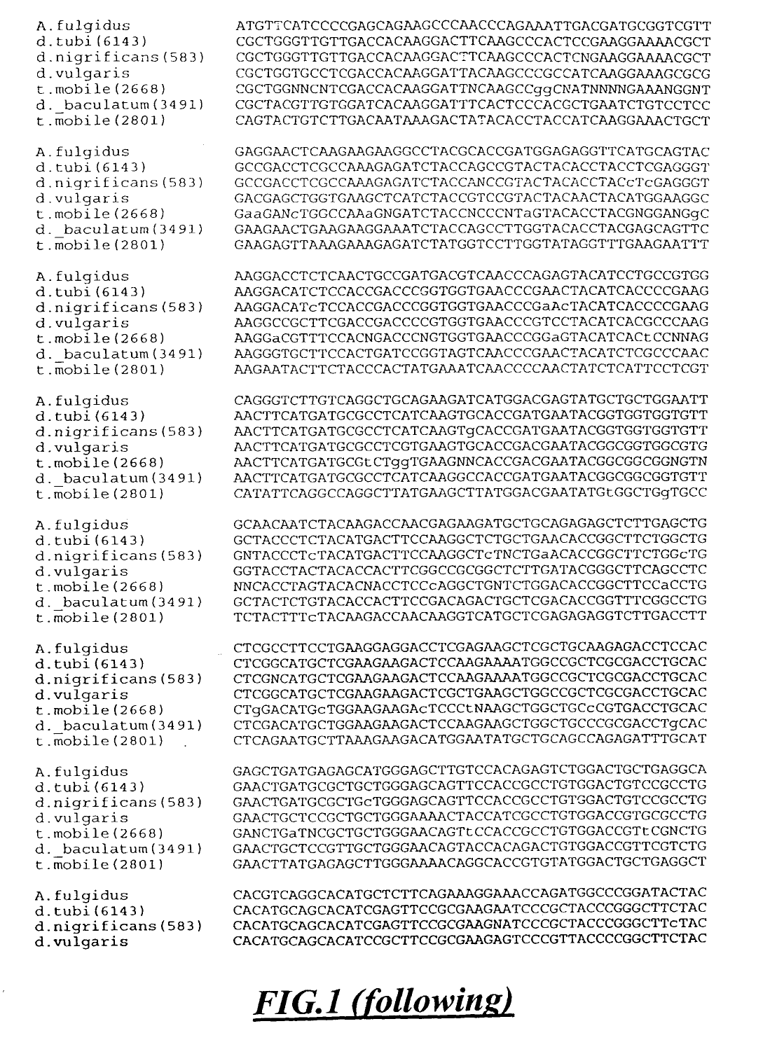

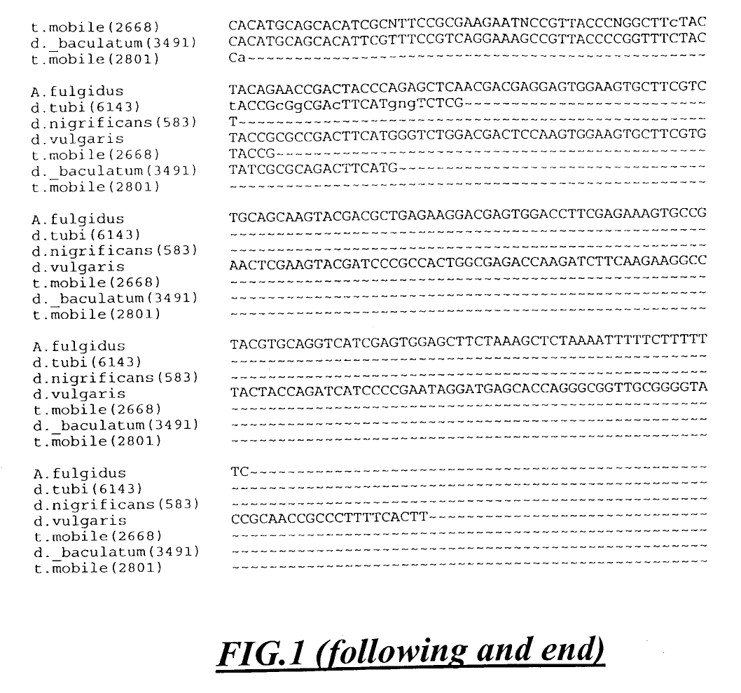

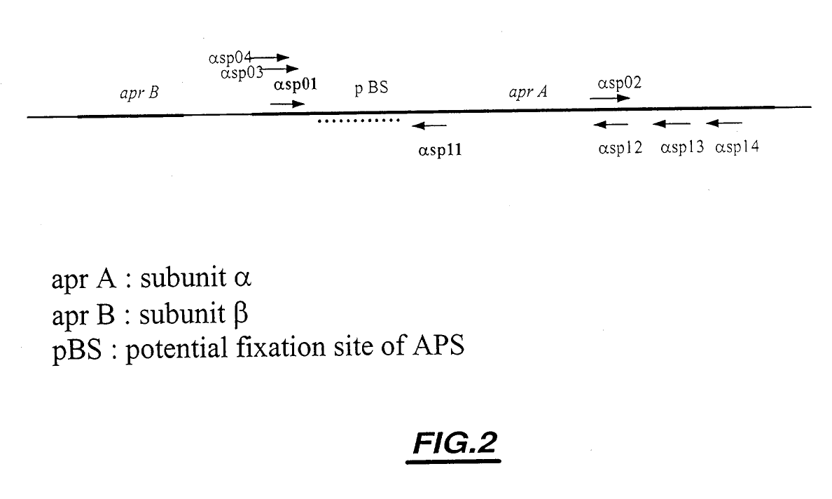

The sequences of the Desulfovibrio vulgaris and Archaeoglobus fulgidus APS reductase genes available in the data banks were aligned. In the regions of homology detected, 10 oligonucleotide sequences capable of allowing the amplification of fragments of the α subunit of APS reductase were defined (cf. Table 1). A fragment of 1580 bp of the Desulfovibrio “tubi” and Desulfomicrobium baculatum APS reductase gene was able to be amplified using the primers APS02 and APS12. In Desulfotomaculum nigrificans and the two strains of Thermodesulfobacterium mobile (SRL2668 and SRL2801), this same fragment was amplified, in two stages, with the aid of the pairs of primers APS04-APS12 and APS02-APS13. The nucleotide sequence of the amplified fragments was determined. The multiple alignment of these sequences, as well as of the reference sequences, is given in FIG. 1. This alignment shows that this gene is surprisingly well conserved in the sulphate-reducing bacteria of the Bacteria domain although the microorganisms studied are phylogenetically distant. This result suggests the potential use of this gene in the context of the detection of SRBs with the aid of molecular diagnostic methods. The authors of the present invention therefore, in order to validate this hypothesis, undertook the construction of new primers in some regions of the gene for the α subunit of APS reductase. These primers, which are located in FIG. 2, are presented in Table 3.

TABLE 3
Primers specific for the α subunit of APS reductase

| SEQ ID No. | Name of the primer | Oligonucleotide sequence | Position D. vulgaris | Orientation |
|---|---|---|---|---|
| 11 | αsp01 | 5′-GATGGAAAACCGCTTCG-3′ | 1575-2591 | Sense |
| 12 | αsp02 | 5′-AAGTTCTCCTCCGGTTC-3′ | 2164-2180 | Sense |
| 13 | αsp03 | 5′-ACCATGATGGAAAACCG-3′ | 1570-1586 | Semse |
| 14 | αsp04 | 5′-TGACCATGATGGAAAAC-3′ | 1568-1584 | Sense |
| 15 | αsp11 | 5′-CGAAGCATCATGTGGTT-3′ | 1765-1781 | Antisense |
| 16 | αsp12 | 5′-CCGGAGGAGAACTTGTG-3′ | 2161-2177 | Antisense |
| 17 | αsp13 | 5′-AGGCGCATCATGAAGTT-3′ | 2371-2367 | Antisense |
| 18 | αsp14 | 5′-ATGTGCTGCATGTGCAG-3′ | 2572-2588 | Antisense |

The various sets of primers thus constructed were tested on various genomic DNAs of sulphate-reducing (SRL 2668, 2801, 2840, 3096, 3491, 3B51, 4225, 6143 and 7861) and non-sulphate-reducing (SRL 3138, 4207, and 4208) bacteria. All the sets of primers tested, with the exception of the αsp02-αsp14 set which does not allow amplification of the APS reductase of Thermodesulfobacterium mobile under the teat conditions, allow a specific amplification of this gene in the SRBs.

The set of primers αsp01-αsp11, which leads to the amplification of a fragment of 205 base pairs (bp), was selected for a control screening on a large number of strains for the following reasons:

very good amplification yield

very little aspecificity (amplification of fragments which are not specific for the chosen target, often of different size; FIG. 3a, line 2, lanes 3 and 4)

primers delimiting the visibly well conserved potential binding site of APS (Speich et al., 1994), which ought to facilitate the construction of other oligonucleotides allowing the specific detection of the amplification products or of the gene itself.

### EXAMPLE 2

Validation of the reference strains for the specificity of the primers αsp01-αsp11. Test for nucleic probes capable of allowing the visualization of the amplification products and definition of the detection threshold under field conditions.

a) Introduction.

The authors of the present invention then showed that the pair of primers selected makes it possible specifically to amplify an oligonucleotide sequence of 205 bp present only in the sulphate reducing bacteria. In addition, based on new nucleic sequences of the fragment of 205 bp, it was possible to define a set of probes which make it possible to visualize, for example, the specific amplification of the SRBs by hybridization. The results of a study aimed at evaluating the sensitivity threshold of the proposed method from a standardized suspension of Desulfovibrio “tubi” (SRL 6143).

b) Materials and Methods

1)—Strains used. In addition to the strains cited in Example 1, the strains specifically used in this section are stated in Table 4, which is presented in section c) of this example.

2)—DNA extractions. Two nucleic acid extraction lo methods were used, one for the preparation of the DNAs of pure strains, the other for the preparation of nucleic acids from field samples. The latter method of extraction is that recommended for the kit itself and is used for the study presented in Example 3.

The extraction of the DNAs from pure strains is carried out using the kit: QIAamp Tissue Kit (QIAGEN, Hilden, Germany) according to the supplier's protocol.

The extraction of the nucleic acids for the detection of the SRBs in samples is carried out as follows: after centrifugation of 1 ml of sample for 30 min at 15,000 rpm, the supernatant is removed. 200 ii of InstaGene™ template (Bio-rad laboratories, Hercules, Calif.) (previously homogenized) are added to the pellet, the mixture is vortexed and incubated for 30 min at 56°0 C. The mixture is vortexed and placed for 8 min at 100° C. The sample is then centrifuged for 2 min at 12,000 rpm, it being possible for 20 μl of supernatant to be used directly in the PCR reactions.

The remainder of the supernatant will be frozen if necessary

3)—PCR Reactions.

The composition of the reaction medium is presented in Table 2.

PCR Programme

| | |
|---|---|
| 1 cycle: | 3 min 95° C. |
| 5 cycles: | 1 min 94° C. |
| | 30 s 54° C. |
| | 10 s 72° C. |
| 35 cycles: | 30 s 94° C. |
| | 30 s 54° C. |
| | 10 s 72° C. |
| 1 cycle: | 5 min 72° C. |
| | hold 4° C. |

For the analysis of the amplification products on 2% agarose gel, 5 μl are deposited, 1 μl of Blue (Sambrook et al., 1989) for the pure strains.

4) Southern Blotting

Deposition on 2% agarose gel of 8 μl of amplification product +2 μl Blue (Sambrook et al., 1989) per well

Migration at 100V for 2 h 0 min

EtBr staining, destaining, photography

Treatment of the gel:

Depurination: 0.25 M HCl: 10 min after destaining blue>yellow (rinsing H₂O)

Denaturation: 0.5 M NaOH, 1.5 M. NaCl: 15 min after restaining yellow>blue (rinsing H₂O)

Neutralizing: 0.5 M Tris pH 8, 1.5 M NaCl: 20×10 min

Transfer onto Hybond N+ film for 4 h 30 min at room temperature by capillarity in a 20×SSC solution

Post-transfer treatments:

labelling of the wells

attaching of the DNA to the membrane for 15 min on a Whatman sheet of paper impregnated with 0.4 N NaOH

rinsing 1 min with 5×SSC

Prehybridization 1 h at 42° C. in:

5×SSC (Sambrook et al, 1989)

5× Denhardt's (Sambrook et al., 1989)

0.5 SDS

100 μg/ml of fish sperm DNA (sonicated and denatured) (DNA, MB grade, from fish sperm, Boehringer Mannheim S. A., Meylan. France)

Labelling of the probe with terminal transferase:

Enzymatic reaction:

2 μl of probe at 50 ng/μl

1 μl of TdT mixture

5 μl of [³²P]dCTP, that is 50 μCi

1 μl of terminal transferase (Pharmacia Biotech)

5 1 μl of DTT at 1 mM

Incubation 15 min at 37° C.

Addition of 10 μl of H₂O and 5 μl of 2% SDS-5 mM EDTA

Elimination of the oligonucleotides not 10 incorporated by P6 column (Bio-rad laboratories, Hercules. Calif.)

Hybridization 16 h at 42° C. in prehybridization buffer+labelled probe activity =10⁶ cpm/ml of buffer

Rinsing: 2×SSC 0.1% SDS at room temperature

Washing: 2×SSC 0.1% SDS at room temperature 15 min

| | | |
|---|---|---|
| 2xSSC | 0.1% SDS at 50° C. | 30 min |
| 1xSSC | 0.1% SDS at 50° C. | 30 min |
| 0.1xSSC | 0.1% SDS at 50° C. | 30 min |

Exposure at −80° C. for 5 h 0 min on X-OMAT™ (Kodak, Rochester, N.Y.)

5)—Test of the probes in microplates according to the sandwich hybridization method Microplates were sensitized in a passive manner with the probe Snbsr 2, used as capture probe which is internal to the amplified product (cf. Table 5). After incubating for 16 to 18 hours, the wells are washed and the plates stored at 4° C. The visualization of the amplified products is made possible by a peroxidase-labelled internal revealing probe (the probes Snbsr 6 and Snbsr 7 were tested independently during this study). The protocol recommended in the Probélia™ kits (SANOFI Diagnostics Pasteur) was used for carrying out the hybridization and detection steps.

6)—Sequencing of the amplified nucleic acids. The sequencing of the amplified nucleic acids was carried out directly on the crude PCR products.

7)—Computer processing of the sequences. The Wisconsin Package programmes, version 9.1-unix, Genetics Computer Group (GCG), Madison, Wisc., were used.

c) Results

The specificity of the set of primers αsp01-αsp11 was evaluated on 37 9genomic DNAs of pure strains extracted from sulphate-reducing and non-sulphate-reducing bacteria. The results obtained, which are given in Table 4, show that the pair of primers defined indeed allows specific amplification of a fragment of the APS reductase gene of sulphate-reducing bacteria.

TABLE 4
Evaluation of the specificity of the pair of
primers αsp01-αsp11

| | | | Amplification |
|---|---|---|---|
| | | | of the specific |
| | | | fragment of ASP |
| | Strains | Name | reductase |
| SRB | SRL 4594 | Desulfovibrio | + |
| | | desulfuricans | |
| | SRL 6146 | Desulfovibrio “gracilis” | + |
| | SRL 422 | Desulfovibrio | + |
| | | desulfuricans | |
| | SRL 4596 | Desulfovibrio sp. | + |
| | SRL 3707 | Desulfovibrio | + |
| | | desulfuricans | |
| | SRL 3137 | Desulfovibrio sp. | + |
| | SRL 2811 | Desulfovibrio | + |
| | | “caledoniensis” | |
| | SRL 2976 | Desulfovibrio | + |
| | | desulfuricans | |
| | SRL 1234 | NI* SRB | + |
| | SRL 3688 | Desulfovibrio | + |
| | | desulfuricans | |
| | SRL 3920 | Desulfovibrio | + |
| | | desulfuricans | |
| | SRL 3663 | Desulfovibrio | + |
| | | desulfuricans | |
| | SRL 3698 | Desulfovibrio | + |
| | | desulfuricans | |
| | SRL 2582 | Desulfovibrio longus | + |
| | SRL 2683 | Desulfovibrio | + |
| | | desulfuricans | |
| | SRL 2810 | Desulfovibrio | + |
| | | desulfuricans | |
| | SRL 3664 | Desulfovibrio | + |
| | | desulfuricans | |
| | SRL 3920 | Desulfovibrio | + |
| | | desulfuricans | |
| | SRL 3096 | Desulfomicrobium. | + |
| | | baculatum | |
| | SRL 3709 | Desulfovibrio | + |
| | | desulfuricans | |
| | SRL 3697 | Desulfovibrio | + |
| | | desulfuricans | |
| | SRL 3706 | Desulfovibrio | + |
| | | desulfuricans | |
| | SRL 3101 | Desulfovibrio lonreachii | + |
| | SRL 3664 | Desulfovibrio | + |
| | | desulfuricans | |
| | SRL 3863 | Desulfovibrio “gracilis” | + |
| | SRL 3699 | Desulfovibrio | + |
| | | desulfuricans | |
| | SRL 3685 | Desulfovibrio | + |
| | | desulfuricans | |
| | SRL 3708 | Desulfovibrio | + |
| | | desulfuricans | |
| | SRL 2979 | Desulfohalobium | + |
| | | retbaense | |
| Non-SRB | | | |
| | SRL 4227 | NI* fermentative | − |
| | SRL 2471 | Haloanaerobium | − |
| | | acetoethylicus | |
| | SRL 4226 | Dethiosulfovibrio | − |
| | | peptidovorans | |
| | SRL 4234 | Geotoga subterranea | − |
| | SRL 4224 | Haloanaerobium | − |
| | | congolense | |
| | SRL 4205 | NI* fermentative | − |
| | SRL 618 | Clostridium glycolicum | − |

*NI: non-identified

Definition of the Internal Amplification Control.

To avoid ambiguous interpretation of the negative results of the PCRs (an absence of amplification by PCR may be due to problem of inhibition of the reaction or to the absence of a target), a plasmid (FIG. 4) was constructed. Added in a limiting concentration to the PCR reaction mixture, this plasmid allows the amplification of a DNA fragment of 289 bp, whose sequence is given in FIG. 5, when no specific target is present in the sample Thus, in the example selected, the presence of a fragment of 289 bp, without a fragment of 205 bp, indicates the functioning of the reaction and the absence of a specific target, that is to Bay of sulphate-reducing bacteria, from the sample studied. In addition, the sequence intercalated between the primers αsp01 and αsp11 in the internal control differs by its size but also by its sequence (Leu2 gene) thus making it possible not to confuse the amplification of the internal control with the specific amplification of a fragment of the APS reductase gene whether the PCR analysis is performed on agarose gel or by hybridization In the example selected, the fragment of 255 bp of the Saccharomyces cerevisiae Leu2 gene was chosen, but any nucleotide sequence may be used provided that it has no homology with the fragment of 205 bp of the APS reductase gene.

It will be noted that this plasmid can only be used as internal control during the detection of sulphate-reducing bacteria with the set of primers αsp01-αsp11. Nevertheless, similar controls can be constructed on the same model in order to make it possible to validate the negative results indicating an absence of a target.

Evaluation of the Sensitivity Threshold

A cellular suspension of Desulfovibrio “tubi” at 10⁸ cells/ml was prepared. It was standardized by dilution in liquid medium by the 3-tube most-probable-number method in the optimum culture medium for the strain (Tardy-Jacquenod, 1996). The evaluation of the sensitivity threshold for the gene amplification was carried out by a 10-fold serial dilution of the cellular suspension and extraction of the nucleic acids according to the InstaGene™ method. By subjecting each nucleic acid extract thus obtained to the PCR test, the authors of the present invention were able to show that the detection of 10 bacteria/ml was possible with the set of primers developed in the presence of an internal control which makes it possible to validate the negative results. These experiments were visualized on agarose gel.

Probes for Visualizing the Specific Amplifications.

Five amplification products obtained with the pair of primers αsp01-αsp11 from genomic DNA of Desulfomicrobium baculatum (SRL 3096), Desulfovibrio longreachii (SRL 3101), Desulfovibrio “gracilias” (SRL 6146), Desulfovibrio desulfuricans (SRL 3707) and Thermodesulfobacterium mobile (SRL 2668) were sequenced in order to verify the specificity of the amplifications. The alignment of all the available sequences (FIG. 6) shows that the remarkable conservation of the gene in this region makes it possible to define 7 new oligonucleotide sequences which can be potentially used for the detection of the gene, or of a fragment of this gene by hybridization or gene amplification (Tab. 5 and FIG. 7).

TABLE 5
Oligonucleotides hybridizing specifically in the fragment
between the primers αsp01 and αsp11 of the α subunit of APS reductase.

| SEQ ID No. | Name | Oligonumcleotide sequence | Position D. vulgaris | Orientation |
|---|---|---|---|---|
| 19 | Snbsr1 | 5′-GACGGTTACGGHCCKGTYGGYGC-3′ | 1609-1631 | Sense |
| 20 | Snbsr2 | 5′-GACGGTTACGGHCCKGTYGGYGCNTGGTTCCT-3′ | 1609-1640 | Sense |
| 21 | Snbsr3 | 5′-GGHCCKGTYGGYGCNTGGTTCCT-3′ | 1618-1640 | Sense |
| 22 | Snbsr4 | 5′-TGGTTCCTKCTSTTCAARGCBAA-3′ | 1630-1655 | Sense |
| 23 | Snbsr5 | 5′-CTSTTCAARGCBAARGCYACCAAC-3′ | 1642-1665 | Sense |
| 24 | Snbsr6 | 5′-AACCGCGCVATGCTSAARCCYTACGA-3′ | 1693-1718 | Sense |
| 25 | Snbsr7 | 5′-TACGARGAWCGCGGHTACGCMAAGGG-3′ | 1714-1739 | Sense |

where
N: A, C, G or T
H: A, C or T
B: G, C or T
V: A, C or G
Y: C or T
M: A or C
W: A or T
K: G or T
S: C or G
R: A or G

These oligonucleotides were tested as probes for the detection of the amplification product of 205 bp by membrane hybridization (Southern blotting). Amplification products obtained with the pair of primers αsp01-αsp11 from genomic DNA of pure strains of sulphate-reducing bacteria (SPL 4225, SRL 6143, SRL 3851, SRL 3492, SRL 2666, SRL 2801) or non-sulphate-reducing bacteria (SRL 4208, SRL 4207, SRL 4226, SRL 6459, SRL 5268, SRL 7311, SRL 3138, SRL 4224) were used in this study.

The results of the hybridization experiments with the probe Snbsr 3 are summarized in Table 6 and are illustrated in FIGS. 3a and 3b.

TABLE 6
Test of the probes for visualizing the
amplification products on membranes (Southern blotting)

| Probe | Size | Specificity⁽¹⁾ | Sensitivity⁽²⁾ | Remarks |
|---|---|---|---|---|
| Snbsr 1 | 23-mers | OK | OK | |
| Snbsr 2 | 32-mers | OK | OK | capture test |
| Snbsr 3 | 23-mers | OK | OK | |
| Snbsr 4 | 23-mers | OK | | |
| Snbsr 5 | 24-mers | OK | | |
| Snbsr 6 | 26-mers | OK | OK | visualization |
| | | | | test |
| Snbsr 7 | 26-mers | OK | OK | visualization |
| | | | | test |

⁽¹⁾Capacity to hybridize only with the PCR products specific to the SRBs (αsp01-αsp11 fragment) in the α subunit of APS reductase.
⁽²⁾Allows hybridization with all the PCR products specific to the SRBs (αsp01-αsp11 fragment) in the α subunit of APS reductase regardless of the strain of origin.

Example of use of the oligonucleotides defined in this work in a “sandwich” type non-radioactive hybridization system.

On the basis of the results obtained by membrane hybridization, a study designed to evaluate a system for detecting the amplification products in microplates, by a “sandwich” type non-radioactive hybridization technique, was undertaken.

The results presented in Tables 7a, 7b and 7c show that the use of the probes selected (Snbsr 2 with Snbsr 6 or Snbsr 7) is possible in terms of sensitivity and specificity for the detection of amplification products obtained with the pair of primers αsp01-αsp11. However, the pair Snbsr 2-Snbsr 6 will be preferred for its sensitivity (difference in values between the blanks and the positive samples).

TABLE 7a
Results of the hybridization tests in microplates with the probe Snbsr 2 used as
capture probe and Snbsr 6 or Snbsr 7 labelled with peroxidase as revealing
probe. Amplification experiment in the absence of internal control on pure strains

| | | | Snbsr 2-Snbsr 6 | | Snbsr 2-Snbsr 7 | |
|---|---|---|---|---|---|---|
| SRL | | asp01-asp11 amplif. of | OD_{405 nm} | Interpret. | OD_{406 nm} | Interpret. |
| Blanks | | without ampl. | 0.159 | / | 0.045 | / |
| | | without ampl. | 0.174 | / | 0.052 | / |
| | | H₂O | 0.096 | / | 0.018 | / |
| | | H₂O | 0.137 | / | 0.027 | / |
| SBR | 61 | Desulfovibrio “tubi” | >3 | + | 0.353 | + |
| | 43 | Desulfovibrio | >3 | + | 2.223 | + |
| | | “bastinii” | | | | |
| | 42 | Desulfomicrobium | 2.150 | + | 1.053 | + |
| | | baculatum | | | | |
| | 25 | Desulfomicrobium | 0.562 | + | 0.211 | + |
| | | baculatum | | | | |
| | 38 | Desulfotomaculum | >3 | + | 1.773 | + |
| | | nigrifiana | | | | |
| | 51 | Thermodesulfobacterium | 0.963 | + | 0.586 | + |
| | | mobile | | | | |
| | 34 | Thermodesulfobacterium | 1.570 | + | 0.422 | + |
| | | mobile | | | | |
| SRB | 3 | | | | | |
| | 26 | | | | | |
| | 68 | | | | | |
| | 28 | | | | | |
| | 01 | | | | | |
| Non- | 64 | Thermatoga elfii | 0.117 | − | 0.025 | − |
| SRB | | | | | | |
| | 59 | NI′ fermentative | 0.139 | − | 0.032 | − |
| | 42 | Dethiosulfovibrio | 0.094 | − | 0.019 | − |
| | | peptidovorans | | | | |
| | 08 | Thermoanaerobacter | 0.098 | − | 0.017 | − |
| | | brockii subsp. | | | | |
| | | lactiethylicus | | | | |
| | 42 | Thermatoga maritima | 0.099 | − | 0.021 | − |
| | 07 | Haloanaerobium | 0.106 | − | 0.014 | − |
| | | congolense | | | | |
| | 52 | Thiobacillus | 0.181 | − | 0.041 | − |
| | | ferroxydans | | | | |
| | 68 | | | | | |
| | 31 | | | | | |
| | 38 | | | | | |
| | 42 | | | | | |
| | 24 | | | | | |
| | 78 | | | | | |
| | 64 | | | | | |

′NI: non identified

TABLE 7b
Results of the hybridization tests in microplates with the probe Snbsr 2 used as
capture probe and Snbsr 6 or Snbsr 7 labelled with peroxidase as revealing
probe. Amplification experiment in the presence of internal control on pure strains

| | | Snbsr 2-Snbsr 6 | | Snbsr 2-Snbsr 7 | |
|---|---|---|---|---|---|
| SRL | asp01-asp11 amplif. of | OD_{405 nm} | Interpret. | OD_{406 nm} | Interpret. |
| Blanks | without ampl. | 0.159 | / | 0.045 | / |
| | without ampl. | 0.174 | / | 0.052 | / |
| | H₂O | 0.271 | / | 0.051 | / |
| | H₂O | 0.299 | / | 0.069 | / |
| SBR | Desulfovibrio “tubi” | >3 | + | 0.321 | + |
| | Desulfovibrio | >3 | + | 2.138 | + |
| | “bastinii” | | | | |
| | Desulfomicrobium | 2.28 | + | 1.160 | + |
| | baculatum | | | | |
| | Desulfomicrobium | 0.667 | + | 0.214 | + |
| | baculatum | | | | |
| | Desulfotomaculum | >3 | + | 1.564 | + |
| | nigrifiana | | | | |
| | Thermodesulfobacterium | 1.17 | + | 0.715 | + |
| | mobile | | | | |
| | Thermodesulfobacterium | 0.889 | + | 0.282 | + |
| | mobile | | | | |
| Non-SRB | Thermotoga elfii | 0.246 | − | 0.056 | − |
| | NI′ fermentative | 0.098 | − | 0.037 | − |
| | Dethiosulfovibrio | 0.088 | − | 0.021 | − |
| | peptidovorans | | | | |
| | Thermoanaerobacter | 0.106 | − | 0.020 | − |
| | brockii subsp. | | | | |
| | lactiethylicus | 0.221 | − | 0.025 | − |
| | Thermatoga maritima | 0.039 | − | 0.029 | − |
| | Haloanaerobium | 0.199 | − | 0.042 | − |
| | congolense | | | | |
| | Thiobacillus | | | | |
| | ferroxydans | | | | |

′NI: non identified

TABLE 7c
Results of the hybridization tests in
microplates with the probe Snbsr 2 used as capture
probe and Snbsr 6 or Snbsr 7 labelled with peroxidase
as revealing probe. Amplification experiment in the
presence of internal control on field samples

| | asp01- | SRB/ml | | | | |
|---|---|---|---|---|---|---|
| | asp11 | “Labége | Snbsr 2-Snbsr 6 | | Snbsr 2-Snbsr 7 | |
| | amplif. | BSR ™ | | Inter- | | Inter- |
| SRL | of | kits” | OD_{405 nm} | pret. | OD_{405 nm} | pret. |
| Blanks | without | / | 0.159 | / | 0.045 | / |
| | ampl. | | | | | |
| | without | / | 0.174 | / | 0.052 | / |
| | ampl. | | | | | |
| | H₂O | / | 0.271 | / | 0.051 | / |
| | H₂O | / | 0.299 | / | 0.069 | / |
| Sample | Cm 1 | 2.5 10⁵ | 2.475* | + | 0.663* | + |
| | Cm 2 | >0.3 | 0.088 | − | 0.014 | − |

*measured from a PCR amplification carried out at the 10⁻⁴ dilution of the sample

### EXAMPLE 3

### Correlation of the Results Obtained by the Gene Amplification and Conventional Culture Methods

a) Introduction

The object of this example is to compare the results of the enumeration of the SRBs in the samples by the gene amplification method (using the oligonucleotides defined in this work) and by the reference culture method for the latter, the “Labège BSR™ Kits” (CFG, Orléans, France) were used, these kits constituting the most sensitive tests on the market.

b) Materials and Methods

samples:

Water from aquifers, edgewater and annular fluids from oil wells.

DNA extractions

The extraction of the nucleic acids for the detection of the SRBs from field samples (InstaGene™ method, Bio-rad laboratories, Hercules, Calif.) was carried out as follows: centrifuge 1 ml of sample for 30 min at 15,000 rpm and then remove the supernatant. Add 200 μl of InstaGene™ matrix (previously homogenized) to the pellet, vortex and incubate for 30 min at 56° C.

Vortex and place for 8 min at 100° C. The sample is then centrifuged for 2 min at 12,000 rpm. In the PCR reactions, 20 μl of supernatant are used. The remainder of the supernatant is frozen if necessary.

PCR Reactions for the Detection of the SRBs

For the routine detection of the sulphate-reducing bacteria, an amplification system which makes it possible to limit the risks of contamination of the PCRs due to the extreme sensitivity of this mode of detection can be used. In this work, the “Core kit Plus™” (Boehringer Mannheim S A, Meylan, France) was used. The reaction conditions are given in Table 8.

TABLE 8
PCR reactions for the detection of the SRBs
under field conditions

| | | | Final | |
|---|---|---|---|---|
| Reagents | Volumes | | concentrations | |
| Primer asp01 (20 μM) | 1.25 | μl | 0.5 | μM |
| Primer asp11 (20 μM) | 1.25 | μl | 0.5 | μM |
| dNTP¹ | 1 | μl | 200 | μM |
| 10X¹ PCR buffer, 25 mM | 5 | μl | | |
| MgCl₂ | | | | |
| MgCl₂ (25 mM)¹ | 3 | μl | 1.5 | mM (+2.5 in |
| | | | | buffer) |
| C1 (Mini-prep at 10⁻⁹) | 2 | μl | ? | |
| DNA prep | 20 | μl | | |
| UDG¹ | 1 | μl | 1 | U |
| Taq Dna pol¹ | 0.5 | μl | 2.5 | U |
| H₂O | qs 50 | μl | | |

¹Boehringer Mannheim S.A. reagents (Meylan, France) PCR Core Kit Plus

Gel analysis: 16 μl of PCR products+4 μl Blue (Sambrook et al, 1989)

PCR Enumeration

The enumeration of the SRBs is carried out by 10-fold serial dilution of the nucleic acid extracts, each dilution then being subjected to a PCR reaction. The interpretation of the result, in terms of number of SRB/ml, is given in Table 9 defined on the basis of the sensitivity threshold measured on a standardized cellular suspension of a pure strain (cf. Example 2).

TABLE 9
Principle of the enumeration of the SRBs by
PCR.

| Reading of the | Reading of the | | |
|---|---|---|---|
| specific | specific | | |
| amplification of | amplification of | | |
| the SRBs | the control | Enumeration | |
| Positive up to the dilution: | | | |
| Not diluted | Not applicable | 10 to 10² | SRB/ml |
| 10⁻¹ | Not applicable | 10² to 10³ | SRB/ml |
| 10⁻² | Not applicable | 10³ to 10⁴ | SRB/ml |
| 10⁻³ | Not applicable | 10⁴ to 10⁵ | SRB/ml |
| 10⁻⁴ | Not applicable | 10⁵ to 10⁶ | SRB/ml |
| 10⁻⁵ | Not applicable | 10⁶ to 10⁷ | SRB/ml |
| 10⁻⁶ | Not applicable | >10⁷ | SRB/ml |
| Negative | Positive | <10 | SRB/ml |
| Negative | Positive from 10⁻¹⁽¹⁾ | <100 | SRB/ml |

| | | |
|---|---|---|
| Negative | Negative⁽²⁾ | Anomaly |

¹In this case, the PCR reaction is only inhibited when the sample is not diluted.
²Absence of amplification due to an experimental anomaly

Enumeration by culture: “Labège BSR™ Kits”

The enumerations are carried out according to the manufacturer's protocol (CFG, Orléans, France). The two methods proposed were used according to the samples:

Series of dilutions with 3 tubes (most-probable-number method—MPN) interpreted by the Mc Crady tables.

Single (1tube) 10-fold series dilutions The result obtained is then a “range”.

c) Results

The results obtained on various field samples (obtained from water from aquifers, edgewater or annular fluids from oil wells) are summarized in Tables 10a and 10b.

TABLE 10a
Summary of the tests carried out using field
sample

| | | Results <10 | |
|---|---|---|---|
| | Negative | by culture | Results |
| Total number | results by | and negative | correlate |
| of analyses | the 2 methods | by PCR* | see Tab. 1b |
| 35 | 13 | 6 | 16 |

*Less than the PCR detection threshold

The negative results were validated (specific amplification of the internal control).

TABLE 10b
Correlation between the microbiological and
PCR enumerations

Results in SRB/ml

| Samples | Cultures⁽¹⁾ | PCR⁽²⁾ |
|---|---|---|
| 1 | 2.5 10¹ | <10² |
| 2 | 2.5 10⁵ | 10⁵-10⁶ |
| 3 | 9.5 10⁴ | 10⁴-10⁵ |
| 4 | <0.3 | 10¹-10² |
| 5 | 0.09 | 10²-10³ |
| 6 | <0.3 | 10¹-10² |
| 7 | <0.3 | 10¹-10² |
| 8 | <0.3 | 10¹-10² |
| 9 | 2.5 10² | 10²-10³ |
| 10 | 10¹-10² | 10²-10³ |
| 11 | 10³-10⁴ | 10²-10³ |
| 12 | 10³-10⁴ | 10²-10³ |
| 13 | 10⁵-10⁶ | 10³-10⁴ |
| 14 | 9.5 10⁴ | 10⁴-10⁵ |
| 15 | 9.5 10² | 10²-10³ |
| 16 | 10² | <10² |

⁽¹⁾“Labege BSR ™ Kits”: results obtained after 21 days of incubation.
⁽²⁾Results obtained in 4 hours.

Twenty six samples give a coherent result by the two methods (of which 20 in Table 10a and 6 in Table10b).

the gene amplification method is more sensitive in 6 cases.

The culture method is found to be more sensitive for 4 samples.

It should be noted that when differences were observed, the enumerations obtained most often only differ by a factor of 10, never by more than a factor of 100, and that these differences are partly due to the fact that the interpretation is made on a range of values.

In addition to the value of the technique presented in terms of speed, the correlation studies presented above show that the PCR method applied to the enumeration of sulphate-reducing bacteria according to the method of use presented here leads to an estimation of the sulphate-reducing bacterial load which is quite comparable with that given by the “Labège BSR™ Kits” Moreover, these results confirm the detection threshold estimated on pure strains under field conditions, since the enumerations carried out by PCR assume that the sensitivity of detection is 10 SRB/ml (cf. sensitivity test, Example 2).

The method of the invention allows the detection and enumeration of sulphate-reducing bacteria from field samples It also makes it possible to fulfill the criteria of sensitivity and speed but also of specificity and reliability inherent to the technique. In addition, this method is not subject to any artifact linked to the method itself: no problem of inhibition of the amplification reaction for a critical detection threshold of less than 100 SRB/ml (even when the sample collected is from an installation treated with biocides), no detection of dead bacteria.

### Bibliographic References

API (1982) Recommended Practice for Biological Analysis of Subsurface Injection Waters American Petroleum Institute, Wash., D.C.

Cord-Ruwich R, Kleinitz W & Widdel F (1987) Sulphate-reducing bacteria and their activities in oil production. J. Petrol. Technol. 1: 97-106.

Horacek G. L & Gawel L. J. (1988) New test kit for the rapid detection of SRB in the oil field. Proceedings of the 63rd American Society of Petroleum Engineers, Houston Oct. 2-5, 1988. SPE paper 18199. Society of Petroleum Engineers, Richardson, Tex.

LeGall J. & Fauque C;. (1988) Dissimilatory is reduction of sulfur compounds. In: A. J. Zehnder (ed.) Biology of anaerobic microorganisms, pp 587-639. John Wiley & Sons, New York.

Magot M., Mondell M. L., Ausseur J. & Seureau J. (1988) Detection of sulphate-reducing bacteria. In: C. C. Gaylarde and L. H. G. Morton “Biocorrosion; proceedings of a joint meeting between the Biodeterioration Society and the French Microbial Corrosion Group” pp 37-52. Biodeterioration Society, Kew, UK.

Postgate J. R. (1979) The sulphate-reducing bacteria. Cambridge University Press, London.

Sambrook J., Fritsch E. F. & Maniatis T. (1989) Molecular cloning—A laboratory manual. Second edition. Cold Spring Harbor Laboratory Press, Cold Spring Harbor, N.Y.

Scott P. J. D. & Davies M. (1992) Survey of field kits for sulfate-reducing bacteria. Materials Performance, May 1992, 64-68.

Speich N., Dahl C., Heisig P. Klein A., Lottspeich F., Stetter K. O. & Trüper H. G. (1994). Adenulylsulphate reductase from sulphate-reducing archaeon Archaeoglobus fulgidus: cloning and characterization of the genes and comparison of the enzyme with other iron sulphur flavoproteins. Microbiology 140: 1273-1284.

Tardy-Jacquenod C. (1996) Biodiversity, taxonomy and phylogeny of sulphate-reducing bacteria isolated from oil fields: examples of salty and hot deposits. Doctoral thesis. University of Bordeaux I.

Tatnall R. E., Stanton K. M. & Ebersole R. C. (1988) Testing for the presence of sulfate-reducing bacteria. Materials Performance, August 1988, 71-80.

Widdel, F. (1988) Microbiology and ecology of sulfate- and sulfur-reducing bacteria. In: Zehnder A. J. B. Biology of anaerobic microorganisms pp 469-585. John Wiley & Sons, New York.

1. Method for the detection of sulphate-reducing bacteria in a sample which is likely to contain them, the said method comprising the extraction of the DNA or of the RNA from the said sample and the detection of at least one fragment of the APS reductace gene or at least one fragment of the mRNA transcribed from the APS reductase gene, an indicator of the presence of sulphate-reducing bacteria in the said sample.

2. Method according to claim 1, in which the detection of at least one fragment of the APS reductase gene comprises the specific gene amplification of at least one fragment of the gene for the α subunit of APS reductase.

3. Method according to claim 1, in which the detection of at least one fragment of the APS reductase gene comprises the hybridization of the extracted DNA with a probe which is specific for the said fragment of the gene for the α subunit of APS reductase, the said probe being labelled in a detectable manner.

4. Method according to claim 2, in which the gene amplification products are subjected to hybridization with a probe which is specific for the said fragment of the gene for the α subunit of APS reductase, the said probe being labelled in a detectable manner.

5. Method according to either of claims 2 and 4, in which at least one primer consisting of an oligonucleotide having a nucleotide sequence which is essentially identical to a sequence chosen from the sequences SEQ ID No 1 to 25 is used for the amplification of the APS reductase gene.

6. Method according to either of claims 3 and 4, in which the said probe has a nucleotide sequence which is essentially identical to a sequence chosen from the sequences SEQ ID No. 1 to 25.

7. Method according to any one of claims 2 or 4 to 6, in which the gene amplification is carried out in the presence of a plasmid including the sequences of the primers specific for a fragment of the APS reductase gene which flank a sequence differing from a fragment of the APS reductase gene.

8. Oligonucleotide having a nucleotide sequence which is essentially identical to a sequence chosen from the sequences SEQ ID No. 1 to 25.

9. Plasmid including two sequences specific for a fragment of the APS reductase gene which flank a sequence differing from a fragment of the APS reductase gene.

10. Plasmid according to claim 9, in which the said sequences specific for a fragment of the APS reductase gene are chosen from the sequences which are essentially identical to the sequences SEQ ID No. 1 to 25.

11. Use of at least one nucleotide sequence which hybridizes specifically with a fragment of the APS reductase gene or of the mRNA transcribed from the APS reductase gene to detect the presence of sulphate-reducing bacteria in a sample.
